(19) United States
(12) Patent Application Publication
(10) Pub. No.: US 2024/0055096 A1
(43) Pub. Date: Feb. 15, 2024

(54) METHOD AND APPARATUS FOR GENERATING A CIRCUIT PROTOCOL FOR INSTITUTING A DESIRED BODY MASS INDEX

(71) Applicant: KPN INNOVATIONS, LLC., LAKEWOOD, CO (US)

(72) Inventor: Kenneth Neumann, LAKEWOOD, CO (US)

(73) Assignee: KPN INNOVATIONS, LLC., LAKEWOOD, CO (US)

(21) Appl. No.: 17/884,936

(22) Filed: Aug. 10, 2022

(51) Int. Cl. G16H 20/30; G16H 20/60; G16H 10/60

(52) U.S. Cl. CPC G16H 20/30; G16H 20/60; G16H 10/60

(57) ABSTRACT

In an aspect, a system and method for developing a generating a circuit protocol for instituting a desired body mass index (BMI) including receiving at least a body mass index representation and a circuit record, generating at least a change of mode by receiving training data correlating mode elements to BMI representations, training a machine learning model as a function of a machine learning algorithm and the training data, and generating at least a change of mode as a function of the machine learning model, and the circuit record, and generating the circuit protocol as a function of the at least a change of nutrition.

### FIELD OF THE INVENTION

The present invention generally relates to the field of AI and simulation/modeling. In particular, the present invention is directed to generating a circuit protocol for instituting a desired body mass index.

### BACKGROUND

Existing computational methods fail to generate accurate guidance when faced with multi-faceted data. This is particularly exacerbated where the data is not readily amenable to exact delineation.

### SUMMARY OF THE DISCLOSURE

In an aspect an apparatus for generating a circuit protocol for instituting a desired body mass index includes a processor configured to receive at least a BMI representation and a circuit record, generate, using the circuit record, at least a change of mode, where generating the at least a change of mode, additionally includes receiving training data correlating mode elements to BMI representations, training a machine learning model as a function of a machine learning algorithm and the training data, generating at least a change of mode as a function of the machine learning model and the circuit record, obtain an activity profile, identify a plurality of activity categories, compute a desired increase in activity, establish an activity type and output the circuit record as a function of the at least a change of mode.

In another aspect a method of generating a circuit protocol for instituting a desired body mass index change includes receiving, using a processor, at least a BMI representation and a circuit record, generating, using the processor, and the circuit record, at least a change of mode, where generating the at least a change of mode, additionally includes receiving training data correlating to mode elements to BMI representations, training a machine learning model as a function of a machine learning algorithm and the training data, and generating at least a change of mode as a function of the machine leaning model and the circuit record, obtaining an activity profile, identifying a plurality of activity categories, computing a desired increase in activity, establishing an activity type and outputting, using the processor, the circuit protocol as a function of the at least a change of mode.

These and other aspects and features of non-limiting embodiments of the present invention will become apparent to those skilled in the art upon review of the following description of specific non-limiting embodiments of the invention in conjunction with the accompanying drawings.

### BRIEF DESCRIPTION OF THE DRAWINGS

For the purpose of illustrating the invention, the drawings show aspects of one or more embodiments of the invention. However, it should be understood that the present invention is not limited to the precise arrangements and instrumentalities shown in the drawings, wherein:

FIG. 1 is a block diagram of an exemplary embodiment of a system generating a circuit protocol for instituting a desired body mass index;

FIG. 2 is a block diagram of an exemplary embodiment of a machine-learning module;

FIG. 3 is a schematic diagram of an exemplary embodiment of a neural network;

FIG. 4 is a schematic diagram of an exemplary embodiment of a node of a neural network;

FIG. 5 is a flow diagram of an exemplary embodiment of a method for generating a circuit protocol for instituting a desired body mass index;

FIG. 6 is a schematic diagram illustrating an exemplary embodiment of a fuzzy inferencing system;

FIG. 7 is a block diagram of a computing system that can be used to implement any one or more of the methodologies disclosed herein and any one or more portions thereof.

The drawings are not necessarily to scale and may be illustrated by phantom lines, diagrammatic representations and fragmentary views. In certain instances, details that are not necessary for an understanding of the embodiments or that render other details difficult to perceive may have been omitted.

### DETAILED DESCRIPTION

At a high level, aspects of the present disclosure are directed to an apparatus and methods for generating a circuit protocol for instituting a desired body mass index (BMI) change. In an embodiment, a circuit protocol may be instituted to induce a desired BMI change. In some embodiments, a circuit protocol may include meal plans, exercise plans, and the like that together are likely to institute a desired BMI change.

Aspects of the present disclosure can be used to reliably predict the circuit protocol that may be useful in instituting a desired BMI change. Aspects of the present disclosure can also be used to determine a desired BMI change, by comparing current BMI representation to a standard BMI representation.

Aspects of the present disclosure allow for lifestyle changes to be used to predictable affect BMI changes. Exemplary embodiments illustrating aspects of the present disclosure are described below in the context of several specific examples.

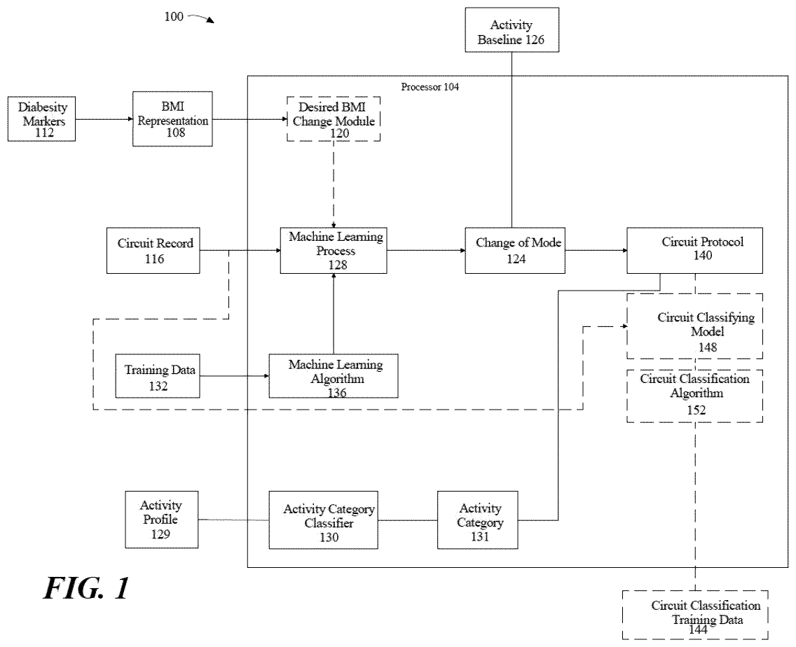

Referring now to FIG. 1, an exemplary embodiment of a system 100 for generating a circuit protocol for instituting a desired BMI change is illustrated. A “desired BMI change” is a change in at least the body mass index within a person, for instance without limitation a change in the body fat percentage. In some cases, a desired BMI changes may include a change to one or more metabolic processes (e.g. usage/breakdown of glucagon, amylin, GIP, GLP-1, epinephrine, glucose, insulin, and the like). In some cases, a desired BMI change is desired to combat diabetes mellitus and obesity. Some non-limiting examples of desired BMI changes that may impact a metabolic process include: a decrease in insulin resistance, increase insulin production, lower blood sugar levels, lower cholesterol levels, and the like. A BMI change may be indicated by at change in at least a biomarker, for instance without limitation a measure level of at least a hormone within a bodily tissue of fluid.

Apparatus includes a processor 104. Processor 104 may include any computing device as described in this disclosure, including without limitation a microcontroller, microprocessor, digital signal processor (DSP) and/or system on a chip (SoC) as described in this disclosure. Computing device may include, be included in, and/or communicate with a mobile device such as a mobile telephone or smartphone. processor 104 may include a single computing device operating independently, or may include two or more computing device operating in concert, in parallel, sequentially or the like; two or more computing devices may be included together in a single computing device or in two or more computing devices. processor 104 may interface or communicate with one or more additional devices as described below in further detail via a network interface device. Network interface device may be utilized for connecting processor 104 to one or more of a variety of networks, and one or more devices. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network may employ a wired and/or a wireless mode of communication. In general, any network topology may be used. Information (e.g., data, software etc.) may be communicated to and/or from a computer and/or a computing device. processor 104 may include but is not limited to, for example, a computing device or cluster of computing devices in a first location and a second computing device or cluster of computing devices in a second location. processor 104 may include one or more computing devices dedicated to data storage, security, distribution of traffic for load balancing, and the like. processor 104 may distribute one or more computing tasks as described below across a plurality of computing devices of computing device, which may operate in parallel, in series, redundantly, or in any other manner used for distribution of tasks or memory between computing devices. processor 104 may be implemented using a “shared nothing” architecture in which data is cached at the worker, in an embodiment, this may enable scalability of system 100 and/or computing device.

With continued reference to FIG. 1, processor 104 may be designed and/or configured to perform any method, method step, or sequence of method steps in any embodiment described in this disclosure, in any order and with any degree of repetition. For instance, processor 104 may be configured to perform a single step or sequence repeatedly until a desired or commanded outcome is achieved; repetition of a step or a sequence of steps may be performed iteratively and/or recursively using outputs of previous repetitions as inputs to subsequent repetitions, aggregating inputs and/or outputs of repetitions to produce an aggregate result, reduction or decrement of one or more variables such as global variables, and/or division of a larger processing task into a set of iteratively addressed smaller processing tasks. processor 104 may perform any step or sequence of steps as described in this disclosure in parallel, such as simultaneously and/or substantially simultaneously performing a step two or more times using two or more parallel threads, processor cores, or the like; division of tasks between parallel threads and/or processes may be performed according to any protocol suitable for division of tasks between it circuits. Persons skilled in the art, upon reviewing the entirety of this disclosure, will be aware of various ways in which steps, sequences of steps, processing tasks, and/or data may be subdivided, shared, or otherwise dealt with using it circuit, recursion, and/or parallel processing.

Still referring to FIG. 1, according to some embodiments, processor 104 is configured to receive at least a BMI representation 108. As used in this disclosure a “BMI representation is a datum indicative of body mass index of a person, for instance without limitation body fat percentage. BMI representation may utilize categories to group ranges of body mass index, such as without limitation: underweight, healthy weight, overweight, and obese. BMI representation may be calculated using a diabesity marker 112. As used in this disclosure a “diabesity marker” is a measurable substance in a human whose presence is indicative of obesity, diabetes mellitus, and the like. Some non-limiting examples of diabesity markers are glucose levels, adipose levels, HbA1c, and the like. Non-limiting examples for testing for diabesity markers 112 may include a fasting glucose test, random glucose test, A1c test, oral glucose tolerance test, and the like.

Continued reference to FIG. 1, testing for diabesity markers 112 may include collecting diabesity data. The term “diabesity data” as used herein, refers to data that indicates the coexistence of both diabetes and obesity. Diabesity data collection may include data stemming from wearable monitoring, surveys, blood samples, electronic monitoring, and the like. Non-limiting examples of wearable diabesity monitoring may include insulin patch-pumps, continuous glucose monitoring systems, and the like. In a non-limiting embodiment of an insulin patch-pump, the patch is temporarily adhered to a human and a small needle directs the insulin into the user's bloodstream. The small needle also may hold the patch in place on the human's body and includes a small cartridge that is filled with prescription, fasting-acting insulin. The physical patch may be paired with a pump which may be computerized device that mimics the way that the human pancreas works by delivering short acting insulin continuously at what may be known as “basal rate.” The pump may connect with the patch to monitor at least a diabesity markers and determine when insulin or other hormones need to be administered to the human user. In a non-limiting embodiment of a continuous glucose monitoring system, a sensor may be inserted under the human user's skin, possibly within the stomach region or arm. The sensor may measure the human user's interstitial glucose level, which may be the glucose found in the fluid between the cells. The sensor tests glucose at set intervals and a transmitter within the sensor wirelessly sends the information to a monitor. The monitor may record information pertaining to at least a diabesity markers. However, the sensor may not deliver medication.

Continuing reference to FIG. 1, non-limiting examples of survey diabesity monitoring may include self-reported use of self-monitoring of at least a diabesity markers, such as blood glucose. Human survey use may utilize a diabesity markers tracking device to manually track, record and submit information pertaining to diabesity markers to their physician.

Still referencing FIG. 1, non-limiting examples of blood sample diabesity monitoring may include fasting glucose test, random glucose test, A1c test, and the like. In a non-limiting embodiment of a fasting glucose test, a human user fasts overnight and then a physician draws blood from the human user to measure a fasting glucose (blood sugar) level on an empty stomach. A fasting glucose level of 99 mg/dL (milligram/deciliter) or lower is considered standard, 100 to 125 mg/dL indicates prediabetes, and 126 mg/dL or higher indicates diabetes. In a non-limiting embodiment of a random glucose test, a human user's blood is measured at the time of testing. The test may happen at any point with or without prior fasting. A glucose (blood sugar) level over 200 mg/dL indicates the human user has diabetes. In a non-limiting example of an A1c test, a human user gives blood that provides information about the average levels of at least a diabesity markers. such as blood glucose, over the past three months. In a non-limiting embodiment of the A1c test, the amount of hemoglobin with attached glucose is measured to reflect the average blood glucose levels.

Referring to FIG. 1, non-limiting examples of electronic monitoring may include flash glucose monitoring, continuous glucose monitoring as described above, and the like. In a non-limiting embodiment of flash glucose monitoring, a sensor may be worn on the back of the human user's arm. The sensor continuously measures the glucose content circuit of the human user's interstitial fluid and alerts the human user when at least a diabesity markers needs attention.

With reference to FIG. 1, processor 104 may be configured to receive circuit record 116. A “circuit record” as used herein, refers to the schedule of a person. A circuit record may include daily schedules, monthly schedules, and the like. Circuit record 116 may comprise of modes. As used herein, “modes” are habits, patterns, customs, and the like. Circuit record 116 may provide data pertaining to eating modes, exercise routines, smoking modes, glucose tracking modes, digestion modes, and the like. Mode modes can indicate contributory factors to diabesity. A mode diet consistent with sustenance that is high in fat, calories, cholesterol, and the like increases risk factors associated with diabesity. Sustenance that is high in calories and fat creates a rise diabesity markers 112, such as blood glucose. Exercise routines, such as strength training, aerobic training. anaerobic training, and the like or lack of exercise routine may affect the human's sensitivity to at least a diabesity markers 112, such as insulin, which is a contributory factor to diabesity. Smoking modes such as habitual cigarette, nicotine vaping, and the like use may decrease sensitivity to at least a diabesity markers 112, such as insulin. Smoking may increase inflammation in the body and cause oxidative stress which may be linked to damaged cells. Smoking also may be linked to a higher risk of abdominal obesity which is a known risk factor for diabesity since abdominal obesity encourages the production of at least a diabesity markers 112, such as cortisol. Glucose tracking modes can indicate how consistent a human user is with monitoring potential fluctuations of at least a diabesity markers 112. Digestion modes such as nausea, heartburn, bloating and the like may indicate fluctuations of at least a diabesity markers 112, such as high glucose levels. A non-limiting example of a digestion issue that may be linked with diabesity is gastroparesis. Gastroparesis is linked to nerve damage within the digestive track that stems from high glucose levels which can lead stomach muscle contractions to slow down or not work at all. If a human user's stomach is not able to empty properly, sustenance can take a long time to leave which affects how fast the body can absorb sustenance and match insulin doses to sustenance portions. Any mode or circuit record 116 that contributes to any links with at least a diabesity markers 112, may be received by processor 104 so processor 104 may provide a robust analysis of the information received.

Referencing FIG. 1, in some embodiments, processor 104 is configured to calculate a desired BMI change as a function of BMI representation 108. For example, in some embodiments, a desired BMI change module 120 may be used to calculate desired BMI changed as a function of BMI representation 108, which may be implemented in any manner suitable for implementation of any computing device, module, and/or component of processor 104 as described above. Modules and/or components described as included in BMI representation 108 are presented for exemplary purposes only; functions and/or structure pertaining to each such module and/or component, module, and/or device incorporated in or communicatively connected to processor 104, in any manner that may occur to persons skilled in the art, upon reviewing the entirety of this disclosure. In some cases, calculated a desired BMI change as a function of BMI representation may include comparing a BMI representation with a BMI standard. Without limitation, a BMI standard may include a normal range of BMI representation. In some cases, a normal range may include a biological reference range having an upper limit and a lower limit; the biological reference range may be based upon measurements from a group of otherwise healthy people. In some cases, normal range may be dependent upon one or more factors, including without limitation age, height, and sex. For example, in some cases a BMI representation may include a measurable metric and a BMI standard may include a normal range, within which the measurable metric is substantially considered unremarkable. Therefore, in some exemplary embodiments, calculating a desired BMI change may additionally include calculating a distance between BMI representation 108 and a BMI standard. A “distance,” as used in this disclosure, is a quantitative value indicating a degree of similarity of a seat of data values to another set of data values. In some cases, a distance between any two or more metrics, for example an BMI representation 108 and a BMI standard or circuit standard and at least a circuit element, may be calculated using any method described in detail below.

Still referring to FIG. 1, for instance, and without limitation, a BMI representation, and a BMI standard, may be represented a vector. Each vector may be represented, without limitation, as an n-tuple of values, where n is at least two values. Each value of n-tuple of values may represent a measurement or other quantitative value associated with a given category of data, or attribute, such as a BMI measure, examples of which are provided in further detail throughout this disclosure; a vector may be represented, without limitation, in n-dimensional space using an axis per category of value represented in n-tuple of values, such that a vector has a geometric direction characterizing the relative quantities of attributes in the n-tuple as compared to each other. A non-limiting distance may include a degree of vector similarity. Two vectors may be considered equivalent where their directions, and/or the relative quantities of values within each vector as compared to each other, are the same; thus, as a non-limiting example, a vector represented as [5, 10, 15] may be treated as equivalent, for purposes of this disclosure, as a vector represented as [1, 2, 3]. Vectors may be more similar where their directions are more similar, and more different where their directions are more divergent, for instance as measured using cosine similarity; however, vector similarity may alternatively or additionally be determined using averages of similarities between like attributes, or any other measure of similarity suitable for any n-tuple of values, or aggregation of numerical similarity measures for the purposes of loss functions as described in further detail below. Any vectors as described herein may be scaled, such that each vector represents each attribute along an equivalent scale of values. Each vector may be “normalized,” or divided by a “length” attribute, such as a length attribute l as derived using a Pythagorean norm:

$$l = \sqrt{{\sum }_{i = 0}^{n} {{a}_{i}}^{2}} ,$$

where aᵢ is attribute number i of the vector. Scaling and/or normalization may function to make vector comparison independent of absolute quantities of attributes, while preserving any dependency on similarity of attributes; this may, for instance, be advantageous where cases represented in training data are represented by different quantities of samples, which may result in proportionally equivalent vectors with divergent values. As a non-limiting illustration, a BMI standard, and/or one or more subsets thereof, may be represented using a vector or other data structure, and a plurality of BMI representation output from one or more machine-learning processes may be represented by a like data structure, such as another vector; a distance comparing the two data structures may then be calculated and compared to distances calculations to find a minimal distance calculation and/or a set of minimal distance calculations. A set of minimal distance calculations may be a set of distance calculations less than a preconfigured threshold distance from data structure representing a desired BMI function. In some cases, one or more machine-learning processes are utilized to prepare plurality of BMI representations, using a plurality of human user inputs, for example without limitation modes, circuit protocols, and/or at least a circuit element. Preconfigured threshold may be set by one or more expert users and/or determined statistically, for instance by finding a top quartile and/or number of percentiles of proximity in a series of distance determinations over time for user, at one time for a plurality of users, and/or over time for a plurality of users. Plurality of users may include a plurality of users selected by a user classifier, which may classify user to a plurality of users having similar physiological data and/or user data; implementation of a user classifier may be performed, without limitation, as described in U.S. Nonprovisional application Ser. No. 16/865,740, filed on May 4, 2020 and entitled “METHODS AND SYSTEMS FOR SYSTEM FOR MODE RECOMMENDATION USING ARTIFICIAL INTELLIGENCE ANALYSIS OF IMMUNE IMPACTS,” the entirety of which is incorporated herein by reference.

Still referring to FIG. 1, distance may be determined using a distance of and/or used in a classifier. A classifier used to compute distance may include, without limitation, a classifier using a K-nearest neighbors (KNN) algorithm. A “K-nearest neighbors algorithm” as used in this disclosure, includes a classification method that utilizes feature similarity to analyze how closely out-of-sample-features resemble training data to classify input data to one or more clusters and/or categories of features as represented in training data; this may be performed by representing both training data and input data in vector forms, and using one or more measures of vector similarity to identify classifications within training data, and to determine a classification of input data. K-nearest neighbors algorithm may include specifying a K-value, or a number directing the classifier to select the k most similar entries training data to a given sample, determining the most common classifier of the entries in the database, and classifying the known sample; this may be performed recursively and/or iteratively to generate a classifier that may be used to classify input data as further samples. For instance, an initial set of samples may be performed to cover an initial heuristic and/or “first guess” at an output and/or relationship, which may be seeded, without limitation, using expert input received according to any process as described herein. As a non-limiting example, an initial heuristic may include a ranking of associations between inputs and elements of training data. Heuristic may include selecting some number of highest-ranking associations and/or training data elements. KNN algorithm may operate according to any suitable distance, including without limitation vector similarity as described above.

With continued regards to FIG. 1, computing device 104 may be configured to generate a classifier using a Naïve Bayes classification algorithm. Naïve Bayes classification algorithm generates classifiers by assigning class labels to problem instances, represented as vectors of element values. Class labels are drawn from a finite set. Naïve Bayes classification algorithm may include generating a family of algorithms that assume that the value of a particular element is independent of the value of any other element, given a class variable. Naïve Bayes classification algorithm may be based on Bayes Theorem expressed as P(A/B)=P(B/A) P(A)÷P(B), where P(A/B) is the probability of hypothesis A given data B also known as posterior probability; P(B/A) is the probability of data B given that the hypothesis A was true; P(A) is the probability of hypothesis A being true regardless of data also known as prior probability of A; and P(B) is the probability of the data regardless of the hypothesis. A naïve Bayes algorithm may be generated by first transforming training data into a frequency table. Computing device 104 may then calculate a likelihood table by calculating probabilities of different data entries and classification labels. Computing device 104 may utilize a naïve Bayes equation to calculate a posterior probability for each class. A class containing the highest posterior probability is the outcome of prediction. Naïve Bayes classification algorithm may include a gaussian model that follows a normal distribution. Naïve Bayes classification algorithm may include a multinomial model that is used for discrete counts. Naïve Bayes classification algorithm may include a Bernoulli model that may be utilized when vectors are binary.

Still referring to FIG. 1, in some embodiments, processor 104 is further configured to generate, using a circuit record 116, at least a change of mode 124. “A change of mode” may refer to a change of at least a habit or routine. For example, in some cases, a change of mode may include exercising for at least one hour a day. Alternatively, or additionally, a change of mode 124 may include a change of circuit record 116, for instance quitting smoking. In some cases, one or more machine-learning processes are employed in generating a change of mode 124. For instance, machine learning model 128 may include receiving training data 132 and generating at least a change of mode 124 as a function of machine learning model 128, and the circuit record 116. Machine learning model 128 may refer to any machine learning model described in this disclosure; and a machine learning algorithm may refer to any machine learning algorithm 136 used in this disclosure. Further explanation of machine learning processes can be found below, in detail. Training data 132 is also described in great detail below; and training data 132 may refer to any training data used in this disclosure.

With continued reference to FIG. 1, at least a change of mode 124 further comprises retrieving an activity baseline. An “activity baseline” as used herein, refers to the actions that a user engages with daily. In a non-limiting embodiment, activity baseline may include degree of cardiovascular activity, degree of anerobic activity, and the like. Cardiovascular activity may be defined as any vigorous activity that increases heart rate and respiration and raised oxygen and blood flow throughout the body. Cardiovascular activity may be initiated through aerobic exercise. Aerobic exercise may be physical exercise of low to high intensity, include but not limited to weightlifting, swimming, cycling, walking, rowing, elliptical training, and the like. Anerobic activity may be defined as short, fast, high-intensity exercises that break down glucose in the body's muscles for a form of energy without the use of increased amounts of oxygen. Anerobic activity may have a shorter duration than aerobic activity. Types of anerobic activity may include high-intensity interval training, weightlifting, circuit training, Pilates, yoga and the like. Anerobic exercises may be used to improve cardiovascular endurance as well as build and maintain muscle and lose body fat. Anaerobic exercises may boost metabolism. Degrees of activity baseline activities, such as cardiovascular activity and anerobic activities, may be compared against a standard activity baseline for a “healthy individual.” Cardiovascular activity and anerobic activity degrees may be calculated using an intensity scale with a numeric, alphabetical, and the like range. A low numeric or alphabetical value may represent minimal exertion with no physical signs, where a high numeric or alphabetical value may represent high exertion with heavy sweating and exhaustion. A change in mode may recommend partaking in cardiovascular or anerobic activity that maintains a moderate to high intensity level to maximize the benefits of the activity.

Still referring to FIG. 1, computing device may develop baseline using data received from a user, for instance and without limitation via a graphical user interface, web form, or the like. Alternatively or additionally, information for developing baseline may be received using one or more wearable devices on user and/or one or more imaging and/or video devices. In either case, data for baseline may be received in the form of data indicating one or more motions of a user. For instance, in the case of an image and/or video capture device, user may be identified using one or more image classifiers, which may be implemented in any manner suitable for a classifier as described in this disclosure and may be trained to recognize a human form and/or a specific user. Image classifiers may further be trained and utilized to recognize different motions of user, for instance and without limitation by recording such motions as affine transformations and/or motion vectors describing motion of a user or a body part of user from one location to another as frames progress in a video feed. Classifiers may be used to identify and/or match motions to such motion vectors, affine transformations, or the like. Further classifiers may match motion vectors, affine transformations, or the like to one or more body motions of a plurality of body motions. Training data for any of the above classifiers may be collected by capture of still and/or video images of persons, which may then be labeled by users to identify (a) a user, (b) a body part, (c) a body motion, or (d) a direction and/or magnitude of motion drawn on a screen; alternatively or additionally, motion vectors and/or affine transformations may be calculated by tracking motion of one or more identified points on a user body such as a joint, extremity, or other part of user as identified by an image classifier, and using differences between locations and elapsed time to derive affine motions and/or motion vectors, which may in turn be associated with user labels of particular body motions or sequences thereof.

Further referring to FIG. 1, a wearable on a user may capture one or more movements of user body and/or body parts using accelerometers, gyroscopes, and/or other motion capture devices and/or inertial measurement units. Such movements may be associated with motion vectors and/or affine transformations or the like as above. Movements and/or motion vectors and/or affine transformations or the like may be labeled as particular body motions by, for instance, a person observing and/or performing movements captured by wearable devices; alternatively or additionally, labeling of motion vectors and/or affine transformations or the like in video data as above may be used. Labeling of motion vectors, affine transformations, or the like may be used to train a classifier, as above, that is able to identify particular body motions.

Still referring to FIG. 1, computing device 104 may develop baseline at least in part by determining durations, frequencies, and/or intensities of body motions. Duration and/or frequency may be determined by adding up time measured during motions, enumerating motions such as steps, strokes, or other repetitions, and/or otherwise aggregating motions to identify sustained and/or repeated exercise; sequences of motions may be labeled by persons and used as training data for a motion sequence classifier, which may be used to identify motion sequences as particular forms of exercise, when input sequences of motion. In a non-limiting example, a series of motion sequences over a period such as a day, a week, a month, or longer may be identified and enumerated to establish that user is performing a particular form of exercise habitually, such as jogging, body-weight exercises, resistance exercises such as weight training, or the like.

Further referring to FIG. 1, computing device 104 may determine an intensity level of a body motion and/or sequence of body motions. Intensity level may indicate an amount of energy expended per motion and/or per unit of time; in other words, a given body motion that is performed more vigorously or “explosively” may have a higher level of intensity that a body motion with a lower degree of vigor or explosiveness. As a non-limiting example, jumping 3 feet off the ground may have a higher degree and/or level of intensity than jumping 1 foot off the ground, running 100 meters in 12 seconds may be associated with a higher degree of intensity than a 100-yard dash that takes 20 seconds. As a further example, any action that involves vertical motion, such as without limitation jumping or squatting, may be higher intensity if it involves a greater degree of vertical displacement. Any action that involves acceleration or deceleration may be higher intensity if it involves a greater degree of acceleration. A higher heart rate and/or a heart rate exceeding a resting heart rate by a greater difference may be associated with an exercise at a higher intensity level. Intensity level may be calculated, without limitation, by performing a fast Fourier transform (FFT) of any signal as described above, including motion sensor data and/or video data, and determining an average and/or peak frequency, where higher average or peak frequencies may indicate greater degree of acceleration. Heart rate, vertical displacement, and/or velocity may be measured directly. Muscular effort may be measured, without limitation, by training a machine-learning model to compare patterns of movement to degree of loads; for instance, video and/or motion sensor data for an exercise may be labeled by a person with a weight or resistance thereto, training a classifier and/or scoring algorithm to output a degree of weight or intensity undergone in an exercise given an input body movement or sequence of body movements. Any or all of the above-described calculations may be performed independently and combined to determine an overall degree of intensity; a machine-learning model may, without limitation, be trained using training data combining any of the above inputs with labels entered by users indicating levels of intensity. Such machine-learning model may input inputs as above and output intensity levels. Alternatively or additionally, intensity levels may be entered in a formula or retrieved from a lookup table.

Still referring to FIG. 1, computing device 104 may determine a range of motion of a user from movements and/or motions as described above. A range of motion may be determined by measuring a distance and/or degree of a motion of a limb and/or portion of user's body; a machine-learning model may, without limitation, be trained using training data combining any body motion inputs with labels entered by users indicating a range of motion through which a body part has passed. Such machine-learning model may input inputs as above and output range of motion levels.

With continued reference to FIG. 1, baseline may include without limitation a cardio baseline. A “cardio baseline,” as used herein is a measurement, quantitative field, and/or score that indicates and/or scores an amount and/or intensity of cardiovascular exercise. Aggregations of body motions as described above may be input to a process to calculate cardio baseline directly; alternatively or additionally, habitual exercises determined as described above, for instance and without limitation using a motion sequence classifier, may be entered with numbers indicating duration and/or level of intensity per session. A process for calculating cardio baseline may include, without limitation, use of a cardio baseline machine-learning model, which may input any of the above inputs and output cardio baseline. Computing device may train cardio baseline machine-learning model using training data in which a person labels activities, body motions, habitual actions, or the like with cardio baseline scores and/or amounts. A machine-learning model such as without limitation a regression model and/or neural network may operate to calculate cardio baseline.

With continued reference, to FIG. 1, baseline may include without limitation an intensity baseline. A “intensity baseline,” as used herein is a measurement, quantitative field, and/or score that indicates and/or scores a typical degree of intensity of exercise. Aggregations of body motions as described above may be input to a process to calculate intensity baseline directly; alternatively or additionally, habitual exercises determined as described above, for instance and without limitation using a motion sequence classifier, may be entered with numbers indicating duration and/or level of intensity per session. A process for calculating intensity baseline may include, without limitation, use of an intensity baseline machine-learning model, which may input any of the above inputs and output intensity baseline. Computing device may train intensity baseline machine-learning model using training data in which a person labels activities, body motions, habitual actions, or the like with intensity baseline scores and/or amounts. A machine-learning model such as without limitation a regression model and/or neural network may operate to calculate intensity baseline.

With continued reference, to FIG. 1, baseline may include without limitation a muscularity baseline. A “muscularity baseline,” as used herein is a measurement, quantitative field, and/or score that indicates and/or scores an amount of muscularity in exercise; “muscularity” as used herein is a measure of muscular force and/or energy used in exercises, where a higher degree of muscularity indicates a greater ability to move and/or lift masses and/or bodyweight. For instance, a person capable of bench pressing 400 pounds may have a higher degree of muscularity than a person capable of bench pressing only 200 pounds. Aggregations of body motions as described above may be input to a process to calculate muscularity baseline directly; alternatively or additionally, habitual exercises determined as described above, for instance and without limitation using a motion sequence classifier, may be entered with numbers indicating duration and/or level of intensity per session. A process for calculating muscularity baseline may include, without limitation, use of a muscularity baseline machine-learning model, which may input any of the above inputs and output muscularity baseline. Computing device may train muscularity baseline machine-learning model using training data in which a person labels activities, body motions, habitual actions, or the like with muscularity baseline scores and/or amounts. A machine-learning model such as without limitation a regression model and/or neural network may operate to calculate muscularity baseline.

With continued reference, to FIG. 1, baseline may include without limitation a flexibility baseline. A “flexibility baseline,” as used herein is a measurement, quantitative field, and/or score that indicates and/or scores an amount of flexibility of a user. Aggregations of body motions as described above may be input to a process to calculate flexibility baseline directly; alternatively or additionally, habitual exercises determined as described above, for instance and without limitation using a motion sequence classifier, may be entered with numbers indicating a degree of flexibility required for a given habitual exercise, for instance and without limitation as. A process for calculating flexibility baseline may include, without limitation, use of a flexibility baseline machine-learning model, which may input any of the above inputs and output flexibility baseline. Computing device may train flexibility baseline machine-learning model using training data in which a person labels activities, body motions, habitual actions, range of motion measurements or the like with flexibility baseline scores and/or amounts. Range of motion of body motions may be input to flexibility machine-learning model for various body motions. Alternatively, degree of flexibility associated with such motions may be retrieved from lookup tables indicating a degree of flexibility to be ascribed to a range of motion on a given type of motion, which may be aggregated or otherwise combined to generate an overall baseline. A machine-learning model such as without limitation a regression model and/or neural network may operate to calculate flexibility baseline.

Still referring to FIG. 1, activity, intensity, muscularity, flexibility scores may be fed into an activity level generation system and/or an activity goal system. An activity level generation system may output a current activity level given inputs as above. An activity goal system may generate a desired future activity level using inputs as above and/or an input of an activity level generation system. In some embodiments, a combination of scores may be performed using a weighted combination, such as without limitation a weighted average, according to weights that may be determined using machine-learning; for instance, an activity level machine-learning model may be trained to output an activity level using training data in which a person has associated any of the above inputs with activity levels and/or any of the above categories of inputs with a degree of impact on an activity level. For example, a person such as a doctor or physical trainer may indicate that degree of muscularity is of greater importance, for instance on a scale of 1-10 or other range, than flexibility, or the like. Alternatively or additionally, a fuzzy inferencing system for determination of baseline overall level and/or one or more activity increase goals may be employed, where any or all of cardio, intensity, muscularity, and/or flexibility, or other values measuring degrees or amounts of exercise, may be represented as values and/or fuzzy sets for linguistic variables measuring the same. An inferencing system may use one or more fuzzy inferencing rules as described below to output one or more linguistic variable values and/or defuzzified values indicating current activity level overall or according to categories, and/or goal activity level overall or according to categories.

With continued reference to FIG. 1, processor 104 may be configured to compute a desired increase in activity as a function of the activity baseline. Using data collected from activity baseline, and intensity of activity baseline activities, processor 104 may compute a desired increase in the intensity, consistency, duration, and the like. Computation of a desired increase in activity may employ the use of a traditionally healthy person's activity baseline and compare a traditionally healthy person's activity baseline to the user's activity baseline. The difference in activity baselines may provide the user with a range of increased activity that is needed to obtain the desired increase in activity as a function of the activity baseline. In a non-limiting example, if the user's activity baseline includes approximately 2 hours a week of cardiovascular exercise at a low intensity level and approximately 2 hours a week of anerobic exercise as a medium intensity level and a traditionally healthy person's activity baseline includes approximately 5 hours a week of cardiovascular exercise at a medium intensity level and 2 hours a week of anerobic exercise at a high intensity level, processor 104 may recommend that the user needs to increase the duration and intensity of exercise activity to match the traditionally healthy person's activity baseline. Computing the desired increase in activity may include a gradual increase in activity baseline overtime as opposed to an immediate increase which may be more challenging for the user to sustain. A gradual increase in activity may employ a machine learning process to map out a sequence of changes to the activity profile. The user may submit feedback based on each step of gradual increases to the activity profile 129 which can train the machine learning model to adjust the sequence accordingly. Computing the desired increase in activity as a function of the activity baseline may utilize at least any of the methods described throughout the disclosure. Computing the desired increase in activity as a function of the activity baseline may be consistent with calculating a desired BMI change as a function of the at least a BMI representation.

Continuing to refer to FIG. 1, activity baseline and desired increases in activity baseline computations may include a monitoring system. In a non-limiting embodiment, a monitoring system may be employed to gauge the intensity of the activity baseline activities and measure the increase in intensity of activity baseline activities after a desired increase in activity has been established. A “monitoring system” as used herein, refers to observation and recording of activity baseline activities. Non-limiting examples of methods that may be used to monitor data taken during activity baseline activities may include wearable movement data such as accelerometer, IMIU, step trackers, and the like. Wearable movement data may record bodily responses such as increased heart rates, oxygen intake, and the like. Wearable movement data may indicate if the computed desired increase in activity as a function of the activity baseline is effective. Wearable movement data may also indicate whether the user has been following the recommended activities to increase their activity baseline.

With further reference to FIG. 1, processor 104 may be configured to obtain activity profile. An “activity profile” as used herein, refers to a vocabulary of motions that the user currently engages in. Non-limiting examples of an activity profile 129 may include workout routines, such as running, Pilates, yoga, and the like, walking habits, daily step counts, standing time, and the like. Classifiers may classify motions to profiles; for instance, classifiers may be used to identify body motions, body motion sequences, exercises, intensity levels thereof, or the like. A vector or other enumeration of amounts of different exercises, motions, and/or intensity levels of motions may be generated per user by performing such enumeration and/or aggregation over time. User data may be mapped to a cohort of similar users and/or to a classification label indicating a cohort of similar users; cohorts of users and/or groupings to be labeled may be determined by running a clustering algorithm such as k-means clustering and/or particle swarm optimization to find populations of similar users with regard to motions, exercises, and/or intensity levels. Classification to labels may be performed using any suitable classifier including k-nearest neighbors and/or neural network classifiers. Classification and/or clustering may be performed using any data and/or combinations of data as described above, including without limitation baselines, profiles, and/or any data used in calculation thereof.

In an embodiment, and further referring to FIG. 1, a clustering algorithm may be implemented, as a non-limiting example, using a k-means clustering algorithm. A “k-means clustering algorithm” as used in this disclosure, includes cluster analysis that partitions n observations or unclassified cluster data entries into k clusters in which each observation or unclassified cluster data entry belongs to the cluster with the nearest mean, using, for instance behavioral training set as described above. “Cluster analysis” as used in this disclosure, includes grouping a set of observations or data entries in way that observations or data entries in the same group or cluster are more similar to each other than to those in other groups or clusters. Cluster analysis may be performed by various cluster models that include connectivity models such as hierarchical clustering, centroid models such as k-means, distribution models such as multivariate normal distribution, density models such as density-based spatial clustering of applications with nose (DBSCAN) and ordering points to identify the clustering structure (OPTICS), subspace models such as biclustering, group models, graph-based models such as a clique, signed graph models, neural models, and the like. Cluster analysis may include hard clustering whereby each observation or unclassified cluster data entry belongs to a cluster or not. Cluster analysis may include soft clustering or fuzzy clustering whereby each observation or unclassified cluster data entry belongs to each cluster to a certain degree such as for example a likelihood of belonging to a cluster; for instance, and without limitation, a fuzzy clustering algorithm may be used to identify clustering of gene combinations with multiple disease states, and vice versa. Cluster analysis may include strict partitioning clustering whereby each observation or unclassified cluster data entry belongs to exactly one cluster. Cluster analysis may include strict partitioning clustering with outliers whereby observations or unclassified cluster data entries may belong to no cluster and may be considered outliers. Cluster analysis may include overlapping clustering whereby observations or unclassified cluster data entries may belong to more than one cluster. Cluster analysis may include hierarchical clustering whereby observations or unclassified cluster data entries that belong to a child cluster also belong to a parent cluster.

With continued reference to FIG. 1, computing device may generate a k-means clustering algorithm receiving unclassified physiological state data and outputs a definite number of classified data entry clusters wherein the data entry clusters each contain cluster data entries. K-means algorithm may select a specific number of groups or clusters to output, identified by a variable “k.” Generating a k-means clustering algorithm includes assigning inputs containing unclassified data to a “k-group” or “k-cluster” based on feature similarity. Centroids of k-groups or k-clusters may be utilized to generate classified data entry cluster. K-means clustering algorithm may select and/or be provided “k” variable by calculating k-means clustering algorithm for a range of k values and comparing results. K-means clustering algorithm may compare results across different values of k as the mean distance between cluster data entries and cluster centroid. K-means clustering algorithm may calculate mean distance to a centroid as a function of k value, and the location of where the rate of decrease starts to sharply shift, this may be utilized to select a k value. Centroids of k-groups or k-cluster include a collection of feature values which are utilized to classify data entry clusters containing cluster data entries. K-means clustering algorithm may act to identify clusters of closely related physiological data, which may be provided with user cohort labels; this may, for instance, generate an initial set of user cohort labels from an initial set of user physiological data of a large number of users, and may also, upon subsequent iterations, identify new clusters to be provided new user cohort labels, to which additional user physiological data may be classified, or to which previously used user physiological data may be reclassified.

With continued reference to FIG. 1, generating a k-means clustering algorithm may include generating initial estimates for k centroids which may be randomly generated or randomly selected from unclassified data input. K centroids may be utilized to define one or more clusters. K-means clustering algorithm may assign unclassified data to one or more k-centroids based on the squared Euclidean distance by first performing a data assigned step of unclassified data. K-means clustering algorithm may assign unclassified data to its nearest centroid based on the collection of centroids ci of centroids in set C. Unclassified data may be assigned to a cluster based on [symbol] dist(ci, x)², where argmin includes argument of the minimum, ci includes a collection of centroids in a set C, and dist includes standard Euclidean distance. K-means clustering module may then recompute centroids by taking mean of all cluster data entries assigned to a centroid's cluster. This may be calculated based on ci=1/|Si|Σxi [symbol] Si^{xi}. K-means clustering algorithm may continue to repeat these calculations until a stopping criterion has been satisfied such as when cluster data entries do not change clusters, the sum of the distances have been minimized, and/or some maximum number of iterations has been reached.

Still referring to FIG. 1, k-means clustering algorithm may be configured to calculate a degree of similarity index value. A “degree of similarity index value” as used in this disclosure, includes a distance measurement indicating a measurement between each data entry cluster generated by k-means clustering algorithm and a selected physiological data set. Degree of similarity index value may indicate how close a particular combination of genes, negative behaviors and/or negative behavioral propensities is to being classified by k-means algorithm to a particular cluster. K-means clustering algorithm may evaluate the distances of the combination of genes, negative behaviors and/or negative behavioral propensities to the k-number of clusters output by k-means clustering algorithm. Short distances between a set of physiological data and a cluster may indicate a higher degree of similarity between the set of physiological data and a particular cluster. Longer distances between a set of physiological behavior and a cluster may indicate a lower degree of similarity between a physiological data set and a particular cluster.

With continued reference to FIG. 1, k-means clustering algorithm selects a classified data entry cluster as a function of the degree of similarity index value. In an embodiment, k-means clustering algorithm may select a classified data entry cluster with the smallest degree of similarity index value indicating a high degree of similarity between a physiological data set and the data entry cluster. Alternatively or additionally k-means clustering algorithm may select a plurality of clusters having low degree of similarity index values to physiological data sets, indicative of greater degrees of similarity. Degree of similarity index values may be compared to a threshold number indicating a minimal degree of relatedness suitable for inclusion of a set of physiological data in a cluster, where degree of similarity indices a-n falling under the threshold number may be included as indicative of high degrees of relatedness. The above-described illustration of feature learning using k-means clustering is included for illustrative purposes only, and should not be construed as limiting potential implementation of feature learning algorithms; persons skilled in the art, upon reviewing the entirety of this disclosure, will be aware of various additional or alternative feature learning approaches that may be used consistently with this disclosure.

Activity profile 129 may account for injuries or medical conditions that a user has and the effect that those injuries or medical conditions have on the user's activity profile. Injuries and medical conditions that may impair a user's ability to engage in consistent activity may consist of sprained ankles, back pain, shoulder tears, asthma, blood clotting disorders, arthritis, tendinitis, and the like. Processor 104 may utilize an injury forecaster to identify the degree of injury risk for different activities. An injury forecaster may utilize at least a machine learning process trained with data relating injuries to types of activities performed while the injuries occurred. The injury forecaster may indicate a percentage, degree, level, or the like, chance that an injury may occur while performing that activity. The injury forecaster machine learning process may also be consistent with a supervised machine learning process which may allow a user to input any existing injuries or medical conditions so that the chance of injury can be forecasted utilizing a potentially increased likelihood of injury due to the existing injuries or conditions that a user currently has. In a non-limiting embodiment, circuit record 116 may include activity profile. Since circuit record 116 may encompass the regular routine of a user, this may also include data that is consistent with the data for activity profile.

Continuing reference to FIG. 1, activity profile 129 may include adjacent motions. The term “adjacent” indicates a likely ability to be added to the user's profile without any issues. Adjacent motions may be selected, in part, based on user preferences. Users may incorporate circuit record 116 and describe overall interests, hobbies, and the like and those may be classified to activities having motion vocabularies within a threshold degree of similarity to the user's current profile, but which have at least one additional motion. In a non-limiting embodiment, if a user enjoys yoga, an adjacent motion that may be added to the user's profile could be either hot yoga or Pilates. Hot yoga and Pilates are motions that may be more intense than regular yoga but may not substantially outside of the user's current activity profile. Adding hot yoga or Pilates to the user's activity profile 129 would not change the user's current circuit record but may help achieve a desired increase in activity. Continued reference to FIG. 1, processor 104 may identify a plurality of activity categories as a function of the activity profile. Identification of a plurality of activity categorize may utilize a classification machine-learning process such as an activity category classifier. Activity category classifier 130 may be trained to categorize activities listed within the activity profile 129 to categories of activities based on similarities in the type, style, or intensity of the activity. The term “intensity” refers to the measurable amount of physical exertion used during an activity. Intensity may be measured using levels or stages to represent ranges from low to high physical exertion. Activity category classifier 130 may be trained to add in additional activity recommendations based on the categories of activities that a user currently engages in. The activity category classifier 130 may categorize the activities based on their intensity level, duration, workout style and the like. In a non-limiting embodiment, activity profile 129 may include several cardiovascular activities with high, medium, and low intensities and a few anerobic activities with low to medium intensity. The activity category classifier 130 may classify each of the activities based on their intensity level to display to the user where they can increase activity levels and styles. The activity category classifier 130 may use other user's data in connection with the current user to recommend additional activities to reach their desired increase in activity level. The activity category classifier 130 may utilize a non-disjoint classification system meaning that an activity may fit into at least one activity category 131. Additionally in a non-limiting embodiment, processor 104 may utilize another classifier to identify users in common. Classifiers used to identify users in common could be trained with activity classifier categories and may classify users together based on common activity profiles. Activity category 131 may be used to determine circuit protocol 140. Circuit protocol 140 may utilize activity categories 131 to implement a protocol that may result in a desired BMI change.

Still referring to FIG. 1, recommended exercises and/or adjacent exercises may alternatively or additionally be determined by enumerating successful additions of activities, where a successful addition is associated with sustained activity of the selected type and/or intensity, lack of injury, and/or decrease in diabesity markers, for similar users; similar users may be users classified to user as described above using clustering algorithms and/or classification to labels. Thus, users having a similar baseline and/or profile that attempted a given change in exercise routine and/or new activity may have success and/or frequency of injury assessed, resulting in a predictive score for advisability of a given change in exercise intensity and/or choice of new activity and/or body motion; this may be used to compute likelihood of injury as well. Any or all of these data may be used as training data for a machine-learning process that outputs, e.g., a degree to which an activity is recommended, a degree of risk of injury, and/or an aggregated or combined measure of the two to determine an overall degree of advisability. Candidate changes in activity profile and/or exercise choices may be ordered by such scores and/or presented to user as recommendations.

Activity category classifiers may also be trained using data from at least wearable movement devices which may classify activity categories to intensity levels. Data indicating high physical exertion (high intensity) may be classified in a category for high intensity activities. Wearable movement devices may indicate high intensity activities by indicated raised heart rates, increased oxygen intake, higher blood pressure, and the like. The activities that were performed while utilizing the wearable movement device may be classified to an intensity level by the activity category classifier. These classifications may be used to optimize the activity profile 129 to display the most accurate vocabulary of motions that the user engages in.

With continued reference to FIG. 1, processor 104 may employ user input's regarding changes to their activity profile 129 based on the recommendations indicated by at least the activity category classifier 130. User inputs regarding changes to their activity profile 129 may be reflected in ways of a survey, activity completion log, and the like. User input surveys may use machine learning processes to generate questions based on the recommendations indicated by at least the activity category classifier 130 or any other changes to their activity profile. The machine learning process used to generate survey questions may be trained with activities and relevant categories within the activities. For example, machine learning process used to generate survey questions relating to Pilates may prompt survey questions having to do with Pilates such as, which part of the body was worked, how slow were the movements, was a Pilates Reformer used, and the like. The machine learning process may learn vocabulary relevant to the activity and utilize the learned vocabulary in generated survey questions to verify that the user has engaged with the activity and is learning more about the activity. An activity completion log may be used to determine whether the user's addition of intensity or motions has gone on long enough to become a mode. In a non-limiting embodiment, an activity completion log may prompt a user to fill out a form that identifies the type of workout, level of intensity, current BMI, duration, and the like. The activity completion log may also employ the use of the wearable activity monitors to verify the intensity that an activity was completed with and the duration that an activity lasted. The activity completion log may utilize a machine learning process to determine when an addition of intensity, new activity, and the like has become a mode. Machine learning processes used to determine if an activity has become a mode may be trained with other user data that showcases how many times a new activity or new intensity level was attempted before it became part of that user's daily or habitual practice. Machine learning processes used to determine if an activity has become a mode may use a classifier that uses user surveys or activity completion logs and BMI representations to output whether the addition of intensity or motions has resulted in a desired increase in activity or a desired BMI change. If the machine learning processes used to determine if an activity has become a mode indicates the changes are now part of the user's habitual practice, then processor 104 may redo the activity profile 129 recommendation process to generate new activity levels or styles to bring the user closer to their desired BMI representation change and activity baseline.

In some cases, and with further reference to FIG. 1, training data 132 correlates circuit elements to BMI representations. As used in this disclosure, a “circuit element” is a representation of schedule and/or habit of schedule; for instance, a circuit element may include consumption modes, exercise modes, digestion modes, and the like. Circuit elements as included in training data 132 may refer to any schedules. habits, routines, modes, and the like. BMI representations as included in training data 132 may include any BMI representation of a BMI state, level, balance, change, function, or the like. In some cases, training data 132 associates known relationships between circuits and BMI system function. Known relationships between circuits and BMI representations may be determined, for example from previous intervention of circuit record, previous mode changes, scientific or refereed journal articles, and the like. In some cases, training data 132 may correlate a change of mode to a mode standard and a circuit record. In some embodiments, training data 132 may correlate a change of mode to a mode standard and a circuit record. In some embodiments, processor 104 is additionally configured to generate at least a change of mode 124 by generating at least a change of mode 124 as a function of machine learning model 128, desired BMI change 120. and circuit record 116. For example, without limitation, machine learning model 128 may be configured to accept as input a desired BMI change module 120 and a circuit record 116 and output a change of mode 124. Alternatively, or additionally, in some embodiments, processor 104 is further configured to calculate at least a change of mode as a function of a mode standard and a circuit record. For example, a mode standard may be generated as a function of machine learning model 128 and a BMI standard; the mode standard may, therefore, represent a circuit which if adhered to will result in a normal range of BMI measures. A change of mode 124, may in some embodiments, then be calculated as a function of a mode standard and circuit record 116. Distance between circuit record 116 and mode standard may be performed according to any method for calculating distance or similarity described in this disclosure.

Still referencing FIG. 1, in some embodiments processor 104 is configured to output a circuit protocol 140 as a function of at least a change of mode 124. A “circuit protocol” as used herein, refers to a plan for a schedule change over time, for instance over a predetermined time period, until a desired endpoint has been reached or indefinitely. In some cases, a circuit protocol may include a dietary change, exercise change, or other lifestyle change, such as without limitation, daily exercise routines, meal plans and the like. In some cases, processor 104 may be further configured to output a circuit protocol 140 by receiving circuit classification training data 144, training a circuit classification model 148 as a function of circuit classification algorithm 152 and the circuit classification training data 144, correlating at least a circuit from the circuit record to at least a bin of a plurality of bins, as a function of the circuit classification model 148 and the circuit record 116, selecting a new circuit classified to the at least a bin, as a function of the at least a change of mode 124, and generating a new circuit protocol 140, where the circuit protocol 140 includes the new circuit. In some cases, a new circuit may embody a change of circuit when compared to at least a circuit from the circuit record. In some embodiments, comparison of a new circuit and at least a circuit from the circuit record may be performed by any calculation described within this disclosure, for example without limitation, a distance calculation. In some cases, a circuit classification training data 144 may correlate a plurality of circuits to a plurality of bins. Circuit classification training data 144 may include any training data described throughout this disclosure. Likewise, circuit classifying model 148 and circuit classification algorithm 152 may include any classification models, algorithms, or processes described throughout this disclosure, including but limited to machine-learning processes, classifiers, and the like.

Still referencing FIG. 1, in a non-limiting embodiment, circuit protocol 140 may indicate that changes to gut microbiome composition may influence key factors associated with DIABESITY MARKERS 112 and BMI representation 108. “Gut microbiome” as used herein, refers to the composition of bacteria, fungi and other microbes that help control digestion within a human's gastrointestinal system. Gut microbiota exists symbiotically with the human digestive system. Gut microbiome composition in human's with diabesity may be associated with production or lack of production of DIABESITY MARKERS 112, such as insulin. Humans with diabesity may have lower overall diversity of microbiome composition than humans who are relatively healthy. Non-limiting examples of bacteria that may be lower in diabesity-affected humans are butyrate-producing bacteria, such as class Clostridia and genus Faecalibacterium, nonbutyrate bacteria, such as Haemophilus parainfluenzae, and the like. In a non-limiting example, gut microbiome composition in diabesity patients may be altered using potential transplants of fecal microbiota from a healthy doner. This form of therapy may promote significant weight loss as well as possible regulation of glucose levels. Transplanting a sample of healthy microbiota into a diabesity patient may stimulate the diabesity patient's gastrointestinal system to develop microbiota matching the healthy microbiota that was transplanted into the diabesity patient.

Still referencing FIG. 1, in a non-limiting embodiment, circuit protocol 140 may indicate fat loss will promote the ability to reach the desired BMI change needed to lessen the effects of diabesity. “Fat loss” as used herein, refers to weight loss from fatty tissue on the human body. Fat loss may be achieved through following circuit protocol 140, change of modes 124, and the like.

Still referring to FIG. 1, in some embodiments, processor 104 may be additionally configured to generate at least a change of mode 124 by generating a mode standard as a function of machine learning model 128 and a BMI standard. In some cases, a mode standard by include at least a mode element which is anticipated to result in a desired BMI state (i.e., BMI standard). Processor 104 may be configured to classify the at least a circuit from a circuit record 112 to at least a mode element, as a function of mode classification model and the circuit record. Processor 104 may then be configured to calculate a distance between at least a mode element classified to at least a circuit and mode standard. Distance may be calculated according to any method described throughout this disclosure. Finally, processor 104 may be configured to generate at least a change of mode 124 as a function of distance between mode element and mode standard. In some cases, mode classification model may include a machine-learning model, which may be trained using a training set, such as without limitation mode classification training data. In some cases, classification training data may correlate a plurality of circuits to a plurality of mode elements, for instance without limitation, correlating a food item to mode information about the food item. Mode classification model may be generated by processor 104 as a function of a mode classification algorithm and mode classification training data. In some cases, mode classification algorithm may include any machine-learning process or algorithm described throughout this disclosure.

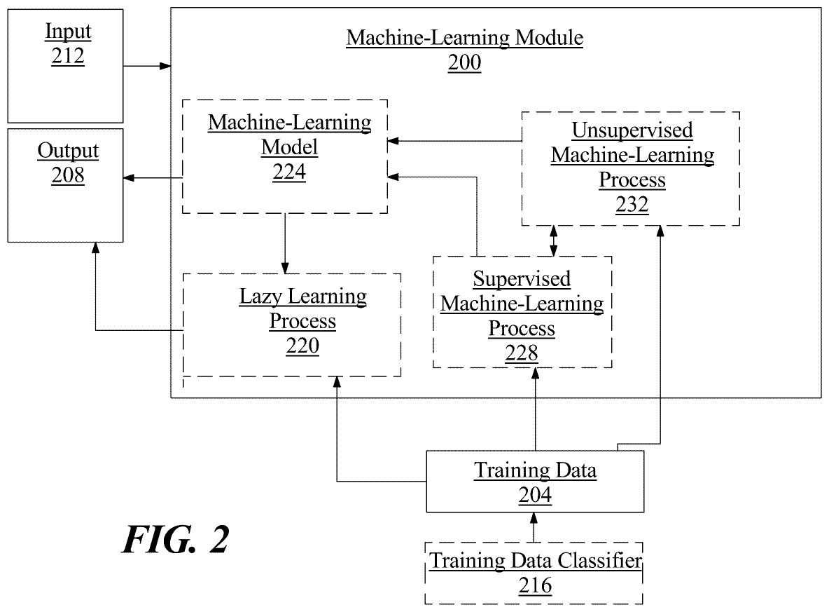

Referring now to FIG. 2, an exemplary embodiment of a machine-learning module 200 that may perform one or more machine-learning processes as described in this disclosure is illustrated. Machine-learning module may perform determinations, classification, and/or analysis steps, methods, processes, or the like as described in this disclosure using machine learning processes. A “machine learning process,” as used in this disclosure, is a process that automatedly uses training data 204 to generate an algorithm that will be performed by a computing device/module to produce outputs 208 given data provided as inputs 212; this is in contrast to a non-machine learning software program where the commands to be executed are determined in advance by a user and written in a programming language.

Still referring to FIG. 2, “training data,” as used herein, is data containing correlations that a machine-learning process may use to model relationships between two or more categories of data elements. For instance, and without limitation, training data 204 may include a plurality of data entries, each entry representing a set of data elements that were recorded, received, and/or generated together; data elements may be correlated by shared existence in a given data entry, by proximity in a given data entry, or the like. Multiple data entries in training data 204 may evince one or more trends in correlations between categories of data elements; for instance, and without limitation, a higher value of a first data element belonging to a first category of data element may tend to correlate to a higher value of a second data element belonging to a second category of data element, indicating a possible proportional or other mathematical relationship linking values belonging to the two categories. Multiple categories of data elements may be related in training data 204 according to various correlations; correlations may indicate causative and/or predictive links between categories of data elements, which may be modeled as relationships such as mathematical relationships by machine-learning processes as described in further detail below. Training data 204 may be formatted and/or organized by categories of data elements, for instance by associating data elements with one or more descriptors corresponding to categories of data elements. As a non-limiting example, training data 204 may include data entered in standardized forms by persons or processes, such that entry of a given data element in a given field in a form may be mapped to one or more descriptors of categories. Elements in training data 204 may be linked to descriptors of categories by tags, tokens, or other data elements; for instance, and without limitation, training data 204 may be provided in fixed-length formats, formats linking positions of data to categories such as comma-separated value (CSV) formats and/or self-describing formats such as extensible markup language (XML), JavaScript Object Notation (JSON), or the like, enabling processes or devices to detect categories of data.

Alternatively, or additionally, and continuing to refer to FIG. 2, training data 204 may include one or more elements that are not categorized; that is, training data 204 may not be formatted or contain descriptors for some elements of data. Machine-learning algorithms and/or other processes may sort training data 204 according to one or more categorizations using, for instance, natural language processing algorithms, tokenization, detection of correlated values in raw data and the like; categories may be generated using correlation and/or other processing algorithms. As a non-limiting example, in a corpus of text, phrases making up a number “n” of compound words, such as nouns modified by other nouns, may be identified according to a statistically significant prevalence of n-grams containing such words in a particular order; such an n-gram may be categorized as an element of language such as a “word” to be tracked similarly to single words, generating a new category as a result of statistical analysis. Similarly, in a data entry including some textual data, a person's name may be identified by reference to a list, dictionary, or other compendium of terms, permitting ad-hoc categorization by machine-learning algorithms, and/or automated association of data in the data entry with descriptors or into a given format. The ability to categorize data entries automatedly may enable the same training data 204 to be made applicable for two or more distinct machine-learning algorithms as described in further detail below. Training data 204 used by machine-learning module 200 may correlate any input data as described in this disclosure to any output data as described in this disclosure. As a non-limiting illustrative example mode element may be correlated to BMI representations, and/or a change of mode may be correlated to a mode standard and a circuit record, and/or a circuit may be correlated to one or more bins.

Further referring to FIG. 2, training data may be filtered, sorted, and/or selected using one or more supervised and/or unsupervised machine-learning processes and/or models as described in further detail below; such models may include without limitation a training data classifier 216. Training data classifier 216 may include a “classifier,” which as used in this disclosure is a machine-learning model as defined below, such as a mathematical model, neural net, or program generated by a machine learning algorithm known as a “classification algorithm,” as described in further detail below, that sorts inputs into categories or bins of data, outputting the categories or bins of data and/or labels associated therewith. A classifier may be configured to output at least a datum that labels or otherwise identifies a set of data that are clustered together, found to be close under a distance metric as described below, or the like. Machine-learning module 200 may generate a classifier using a classification algorithm, defined as a process whereby a computing device and/or any module and/or component operating thereon derives a classifier from training data 204. Classification may be performed using, without limitation, linear classifiers such as without limitation logistic regression and/or naïve Bayes classifiers, nearest neighbor classifiers such as k-nearest neighbors classifiers, support vector machines, least squares support vector machines, fisher's linear discriminant, quadratic classifiers, decision trees, boosted trees, random forest classifiers, learning vector quantization, and/or neural network-based classifiers. As a non-limiting example, training data classifier 216 may classify elements of training data to a plurality of bins. A classifier may include any classifier described throughout this disclosure. In some cases, classification training data may be used to classify at least a circuit to at least a bin (or category), such that circuits may be grouped together by bin. In some cases, bins may be related to a category of circuit for example, vegetable, fruit, starch, meat, fish, and the like. Alternatively, or additionally, bins may be demarcated according to a meal or course at which circuits grouped within them are consumed, for example breakfast, brunch, lunch, dinner, dessert, and the like. Alternatively, or additionally, bins may be demarcated by production type, producer, or circuit originator, for example store or circuit source. Alternatively, or additionally, bins may be demarcated by mode elements, for example in some cases circuits including similar mode elements or like nutrient profiles may be classified together by bin. In some cases, classification may also include generating a probability of classification, for example by way of a Naïve Bayes classification algorithm, as described above.

Still referring to FIG. 2, machine-learning module 200 may be configured to perform a lazy-learning process 220 and/or protocol, which may alternatively be referred to as a “lazy loading” or “call-when-needed” process and/or protocol, may be a process whereby machine learning is conducted upon receipt of an input to be converted to an output, by combining the input and training set to derive the algorithm to be used to produce the output on demand. For instance, an initial set of simulations may be performed to cover an initial heuristic and/or “first guess” at an output and/or relationship. As a non-limiting example, an initial heuristic may include a ranking of associations between inputs and elements of training data 204. Heuristic may include selecting some number of highest-ranking associations and/or training data 204 elements. Lazy learning may implement any suitable lazy learning algorithm, including without limitation a K-nearest neighbors algorithm, a lazy naïve Bayes algorithm, or the like; persons skilled in the art, upon reviewing the entirety of this disclosure, will be aware of various lazy-learning algorithms that may be applied to generate outputs as described in this disclosure, including without limitation lazy learning applications of machine-learning algorithms as described in further detail below.

Alternatively, or additionally, and with continued reference to FIG. 2, machine-learning processes as described in this disclosure may be used to generate machine-learning models 224. A “machine-learning model,” as used in this disclosure, is a mathematical and/or algorithmic representation of a relationship between inputs and outputs, as generated using any machine-learning process including without limitation any process as described above and stored in memory; an input is submitted to a machine-learning model 224 once created, which generates an output based on the relationship that was derived. For instance, and without limitation, a linear regression model, generated using a linear regression algorithm, may compute a linear combination of input data using coefficients derived during machine-learning processes to calculate an output datum. As a further non-limiting example, a machine-learning model 224 may be generated by creating an artificial neural network, such as a convolutional neural network including an input layer of nodes, one or more intermediate layers, and an output layer of nodes. Connections between nodes may be created via the process of “training” the network, in which elements from a training data 204 set are applied to the input nodes, a suitable training algorithm (such as Levenberg-Marquardt, conjugate gradient, simulated annealing, or other algorithms) is then used to adjust the connections and weights between nodes in adjacent layers of the neural network to produce the desired values at the output nodes. This process is sometimes referred to as deep learning.

Still referring to FIG. 2, machine-learning algorithms may include at least a supervised machine-learning process 228. At least a supervised machine-learning process 228, as defined herein, include algorithms that receive a training set relating a number of inputs to a number of outputs, and seek to find one or more mathematical relations relating inputs to outputs, where each of the one or more mathematical relations is optimal according to some criterion specified to the algorithm using some scoring function. For instance, a supervised learning algorithm may include mode elements as described above as inputs, BMI representations as outputs, and a scoring function representing a desired form of relationship to be detected between inputs and outputs; scoring function may, for instance, seek to maximize the probability that a given input and/or combination of elements inputs is associated with a given output to minimize the probability that a given input is not associated with a given output. Scoring function may be expressed as a risk function representing an “expected loss” of an algorithm relating inputs to outputs, where loss is computed as an error function representing a degree to which a prediction generated by the relation is incorrect when compared to a given input-output pair provided in training data 204. Persons skilled in the art, upon reviewing the entirety of this disclosure, will be aware of various possible variations of at least a supervised machine-learning process 228 that may be used to determine relation between inputs and outputs. Supervised machine-learning processes may include classification algorithms as defined above.

Further referring to FIG. 2, machine learning processes may include at least an unsupervised machine-learning processes 232. An unsupervised machine-learning process, as used herein, is a process that derives inferences in datasets without regard to labels; as a result, an unsupervised machine-learning process may be free to discover any structure, relationship, and/or correlation provided in the data. Unsupervised processes may not require a response variable; unsupervised processes may be used to find interesting patterns and/or inferences between variables, to determine a degree of correlation between two or more variables, or the like.

Still referring to FIG. 2, machine-learning module 200 may be designed and configured to create a machine-learning model 224 using techniques for development of linear regression models. Linear regression models may include ordinary least squares regression, which aims to minimize the square of the difference between predicted outcomes and actual outcomes according to an appropriate norm for measuring such a difference (e.g., a vector-space distance norm); coefficients of the resulting linear equation may be modified to improve minimization. Linear regression models may include ridge regression methods, where the function to be minimized includes the least-squares function plus term multiplying the square of each coefficient by a scalar amount to penalize large coefficients. Linear regression models may include least absolute shrinkage and selection operator (LASSO) models, in which ridge regression is combined with multiplying the least-squares term by a factor of 1 divided by double the number of samples. Linear regression models may include a multi-task lasso model wherein the norm applied in the least-squares term of the lasso model is the Frobenius norm amounting to the square root of the sum of squares of all terms. Linear regression models may include the elastic net model, a multi-task elastic net model, a least angle regression model, a LARS lasso model, an orthogonal matching pursuit model, a Bayesian regression model, a logistic regression model, a stochastic gradient descent model, a perceptron model, a passive aggressive algorithm, a robustness regression model, a Huber regression model, or any other suitable model that may occur to persons skilled in the art upon reviewing the entirety of this disclosure. Linear regression models may be generalized in an embodiment to polynomial regression models, whereby a polynomial equation (e.g. a quadratic, cubic or higher-order equation) providing a best predicted output/actual output fit is sought; similar methods to those described above may be applied to minimize error functions, as will be apparent to persons skilled in the art upon reviewing the entirety of this disclosure.

Continuing to refer to FIG. 2, machine-learning algorithms may include, without limitation, linear discriminant analysis. Machine-learning algorithm may include quadratic discriminate analysis. Machine-learning algorithms may include kernel ridge regression. Machine-learning algorithms may include support vector machines, including without limitation support vector classification-based regression processes. Machine-learning algorithms may include stochastic gradient descent algorithms, including classification and regression algorithms based on stochastic gradient descent. Machine-learning algorithms may include nearest neighbors algorithms. Machine-learning algorithms may include Gaussian processes such as Gaussian Process Regression. Machine-learning algorithms may include cross-decomposition algorithms, including partial least squares and/or canonical correlation analysis. Machine-learning algorithms may include naïve Bayes methods. Machine-learning algorithms may include algorithms based on decision trees, such as decision tree classification or regression algorithms. Machine-learning algorithms may include ensemble methods such as bagging meta-estimator, forest of randomized tress, AdaBoost, gradient tree boosting, and/or voting classifier methods. Machine-learning algorithms may include neural net algorithms, including convolutional neural net processes.

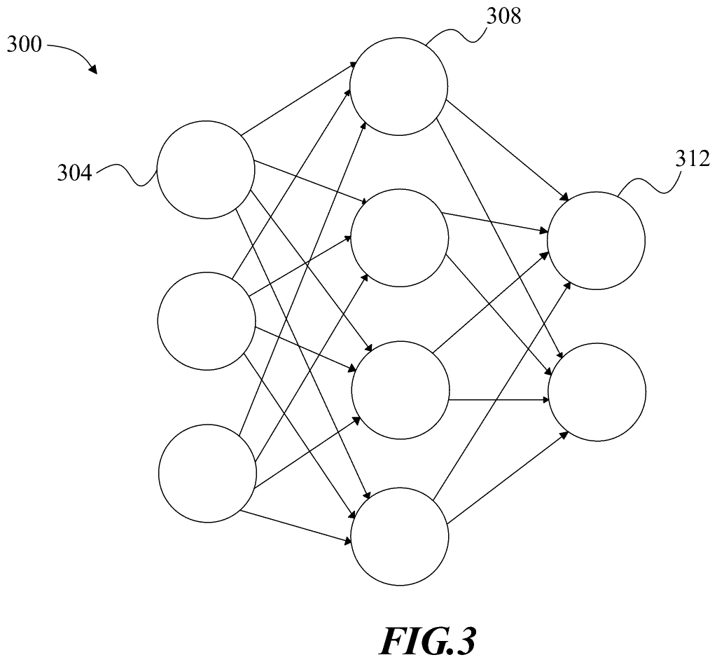

Referring now to FIG. 3 an exemplary embodiment of neural network 300 is illustrated. Neural network also known as an artificial neural network, is a network of “nodes,” or data structures having one or more inputs, one or more outputs, and a function determining outputs based on inputs. Such nodes may be organized in a network, such as without limitation a convolutional neural network, including an input layer of nodes 304, one or more intermediate layers 308, and an output layer of nodes 312. Connections between nodes may be created via the process of “training” the network, in which elements from a training dataset are applied to input nodes 304, a suitable training algorithm (such as Levenberg-Marquardt, conjugate gradient, simulated annealing, or other algorithms) is then used to adjust the connections and weights between nodes in adjacent layers 308 of the neural network to produce the desired values at output nodes 312. This process is sometimes referred to as deep learning.

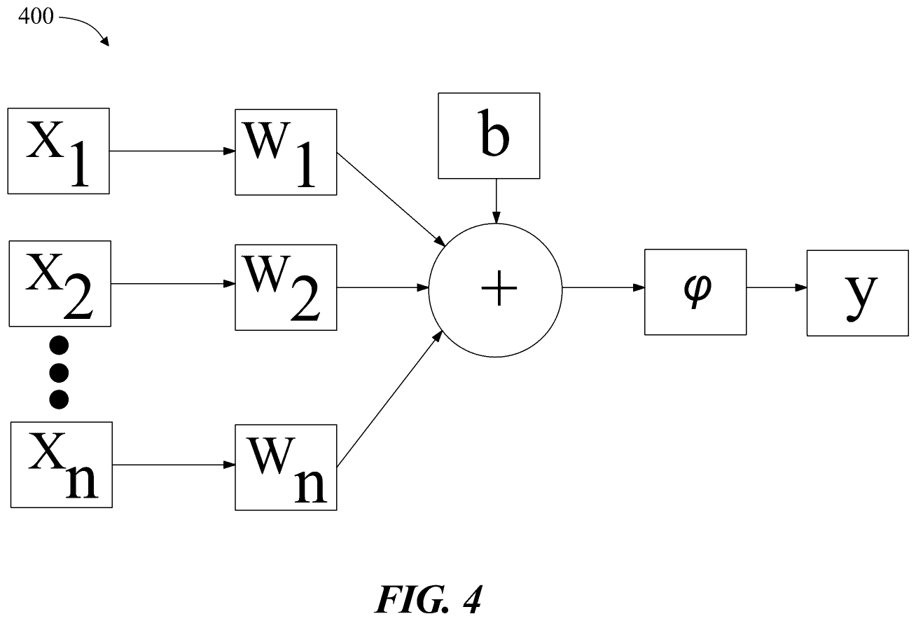

Referring now to FIG. 4, an exemplary embodiment of a node 400 of a neural network is illustrated. A node 400 may include, without limitation a plurality of inputs x; that may receive numerical values from inputs to a neural network containing the node and/or from other nodes. Node 400 may perform a weighted sum of inputs using weights w, that are multiplied by respective inputs xi. Additionally or alternatively, a bias b may be added to the weighted sum of the inputs such that an offset is added to each unit in the neural network layer that is independent of the input to the layer. The weighted sum may then be input into a function p, which may generate one or more outputs y. Weight w, applied to an input x; may indicate whether the input is “excitatory,” indicating that it has strong influence on the one or more outputs y, for instance by the corresponding weight having a large numerical value, and/or a “inhibitory,” indicating it has a weak effect influence on the one more inputs y, for instance by the corresponding weight having a small numerical value. The values of weights w, may be determined by training a neural network using training data, which may be performed using any suitable process as described above.

Still referring to FIG. 4, a neural network may, for example without limitation, receive a desired endocrine change as input and output at least a mode element. Additionally, or alternatively, a neural network may, for example without limitation, at least a mode element as input and output an anticipated BMI change. In some cases, a neural network may, for example without limitation, classify at least a mode element or a change of mode 120 to at least a circuit or a circuit protocol 136. In some cases, a neural network may additionally output a probability of classification to a predetermined class according to weights w, that are derived using machine-learning processes as described in this disclosure. In some cases, a probability of classification may describe a distance between BMI effects anticipated to result from at least a circuit or a circuit protocol 136 and a desired BMI change.

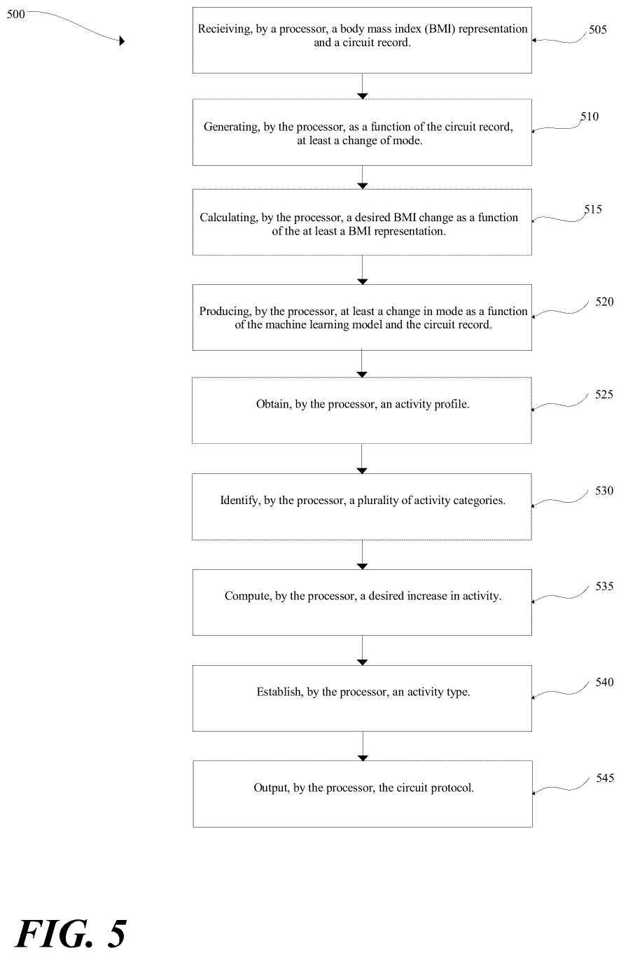

Referring now to FIG. 5, a method 500 of generating a circuit protocol for instituting a desired BMI change is shown by way of a flowchart. At step 505, a processor 104 receives information 505; for instance, without limitation the information may include at least an BMI representation 108 and a circuit record 116. An BMI representation may include any BMI representation described throughout this disclosure, for instance in reference to FIGS. 1-4. A circuit record may include any circuit record described throughout this disclosure, for instance in reference to FIGS. 1-4.

Continuing in reference to FIG. 5, at step 510, a processor 104 generates at least a change of mode 124. In some embodiments, generating a change of mode may additionally include receiving training data correlating mode elements to BMI representations, training a machine learning model as a function of a machine learning algorithm and the training data, and generating the at least a change of mode 124 as a function of the machine learning model, and the circuit record. Change of mode may include any change of mode described throughout this disclosure, for example in reference to FIGS. 1-4. Training data may include any training data described throughout this application, for example in reference to FIGS. 1-4. Machine learning model may include any machine learning model described throughout this disclosure, for example in reference to FIGS. 1-4. Machine learning algorithm may include any machine learning algorithm described throughout this disclosure, for example in reference to FIGS. 1-4. In some versions, step 510 at generating at least a change of mode additionally includes generating a mode standard as a function of a machine learning model and an BMI standard, receiving nutrient classification training data correlating a plurality of circuits to a plurality of mode elements, training a nutrient classification model as a function of a nutrient classification algorithm and the nutrient classification training data, classifying at least a circuit from a circuit record 116 to at least a mode element, as a function of the nutrient classification model and the circuit record, calculating a distance between the circuit record and the mode standard, and generating the at least a change of mode 124 as a function of the distance. In some cases, machine learning model may include a convolutional neural network

Continuing in reference to FIG. 5, at step 515, a processor 104 calculates a desired BMI change. In some cases, desired BMI change may be calculated as a function of an BMI representation 108. Calculating a desired BMI change 515 may be performed according to any calculation methods described throughout this disclosure, including without limitation calculating a distance between an BMI representation and an BMI standard. In some versions, step 510 at calculating desired BMI change may additionally include calculating a distance between the BMI representation and an BMI standard. In some cases, an BMI standard may include a normal range of hormone levels. In some cases, step 515 at calculating distance between BMI representation and an BMI standard additionally includes representing the BMI representation as a first vector, representing the BMI standard as a second vector, calculating a similarity between the first vector and the second vector, and calculating the distance as a function of the similarity between the first vector and the second vector.

Continuing in reference to FIG. 5, at step 520, a processor 104 generates at least a change of mode 124. In some embodiments, generating a change of mode may additionally include receiving training data correlating mode elements to BMI representations, training a machine learning model as a function of a machine learning algorithm and the training data, and generating the at least a change of mode 124 as a function of the machine learning model, and the circuit record. Change of mode may include any change of mode described throughout this disclosure, for example in reference to FIGS. 1-4. Training data may include any training data described throughout this application, for example in reference to FIGS. 1-4. Machine learning model may include any machine learning model described throughout this disclosure, for example in reference to FIGS. 1-4. Machine learning algorithm may include any machine learning algorithm described throughout this disclosure, for example in reference to FIGS. 1-4. In some versions, step 520 at generating at least a change of mode additionally includes generating a mode standard as a function of a machine learning model and an BMI standard, receiving nutrient classification training data correlating a plurality of circuits to a plurality of mode elements, training a nutrient classification model as a function of a nutrient classification algorithm and the nutrient classification training data, classifying at least a circuit from a circuit record 116 to at least a mode element, as a function of the nutrient classification model and the circuit record, calculating a distance between the circuit record and the mode standard, and generating the at least a change of mode 124 as a function of the distance. In some cases, machine learning model may include a convolutional neural network.

Continuing reference to FIG. 5, at step 525, processor 104 obtains an activity profile. An activity profile may include a vocabulary of motions that the user regularly engages with.

Continuing reference to FIG. 5, at step 530, processor 104 identifies a plurality of activity categories as a function of the activity profile. Identifying the plurality of activity categories further comprises an activity category classifier.

Continuing reference to FIG. 5, at step 535, processor 104 computes a desired increase in activity as a function of the activity baseline. The activity baseline of the user may be compared to the activity baseline of a standard healthy person. The desired increase in activity may be implemented through a sequence of gradual increases of intensity of activities.

Continuing reference to FIG. 5, at step 540, processor 104 establishes an activity type as a function of a plurality of activity types, wherein identifying the activity type further comprises an activity type classifier. The activity type classifier may suggest additional activities that a user can engage in to increase their activity levels.

Continuing in reference to FIG. 5, at step 545, a processor 104 outputs a circuit protocol 140. According to some embodiments, circuit protocol may be output as a function of at least a change of mode 124. Circuit protocol may include any circuit protocol described in this disclosure, for example in reference to FIGS. 1-4. In some versions, outputting a circuit protocol additionally includes receiving circuit classification training data correlating a plurality of circuits to a plurality of bins, training a circuit classification model as a function of a circuit classification algorithm and the circuit classification training data, correlating at least a circuit from the circuit record to at least a bin of the plurality of bins, as a function of the circuit classification model and the circuit record, selecting a new circuit classified to the at least a bin, as a function of at least a change of mode, and outputting the circuit protocol, wherein the circuit protocol includes the new circuit. Circuit classification training data may include any training data described throughout this disclosure, for example in reference to FIGS. 1-4. Circuit classification model may include any model, or machine learning model, described throughout this disclosure, for example in reference to FIGS. 1-4. Circuit classification algorithm may include any algorithm, or machine learning algorithm, described throughout this disclosure, for example in reference to FIGS. 1-4. In some versions, classifying the at least a circuit to the at least a bin, additionally includes generating a probability of classification. Probability of classification may include any probability of classification described throughout this disclosure, for example in reference to FIGS. 1-4.

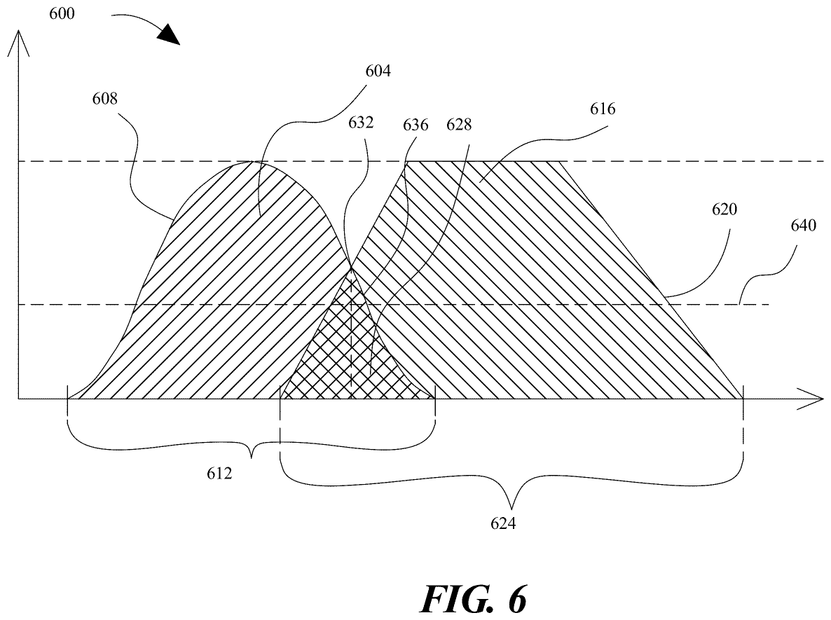

Referring to FIG. 6, an exemplary embodiment of fuzzy set comparison 600 is illustrated. A first fuzzy set 604 may be represented, without limitation, according to a first membership function 608 representing a probability that an input falling on a first range of values 612 is a member of the first fuzzy set 6604, where the first membership function 608 has values on a range of probabilities such as without limitation the interval [0,1], and an area beneath the first membership function 608 may represent a set of values within first fuzzy set 604. Although first range of values 612 is illustrated for clarity in this exemplary depiction as a range on a single number line or axis, first range of values 612 may be defined on two or more dimensions, representing, for instance, a Cartesian product between a plurality of ranges, curves, axes, spaces, dimensions, or the like. First membership function 608 may include any suitable function mapping first range 612 to a probability interval, including without limitation a triangular function defined by two linear elements such as line segments or planes that intersect at or below the top of the probability interval. As a non-limiting example, triangular membership function may be defined as:

$$y ( x , a , b , c ) = \{ \begin{matrix}0 , & for x > c and x < a \\ \frac{x - a}{b - a} , & for a \leq x < b \\ \frac{c - x}{c - b} , & if b < x \leq c\end{matrix}$$

a trapezoidal membership function may be defined as:

$$y ( x , a , b , c , d ) = max ( min ( \frac{x - a}{b - a} , 1 , \frac{d - x}{d - c} ) , 0 )$$

a sigmoidal function may be defined as:

$$y ( x , a , c ) = \frac{1}{1 - {e}^{- a ( x - c )}}$$

a Gaussian membership function may be defined as:

$$y ( x , c , σ ) = {e}^{- \frac{1}{2} {( \frac{x - c}{σ} )}^{2}}$$

and a bell membership function may be defined as:

$$y ( x , a , b , c , ) = {[ 1 + {\vert \frac{x - c}{a} \vert}^{2 b} ]}^{- 1}$$

Persons skilled in the art, upon reviewing the entirety of this disclosure, will be aware of various alternative or additional membership functions that may be used consistently with this disclosure.

Still referring to FIG. 6, first fuzzy set 604 may represent any value or combination of values as described above, including output from one or more machine-learning models and &&&, a predetermined class, such as without limitation of &&& A second fuzzy set 616, which may represent any value which may be represented by first fuzzy set 604, may be defined by a second membership function 620 on a second range 624; second range 624 may be identical and/or overlap with first range 612 and/or may be combined with first range via Cartesian product or the like to generate a mapping permitting evaluation overlap of first fuzzy set 604 and second fuzzy set 616. Where first fuzzy set 604 and second fuzzy set 616 have a region 628 that overlaps, first membership function 608 and second membership function 620 may intersect at a point 662 representing a probability, as defined on probability interval, of a match between first fuzzy set 604 and second fuzzy set 616. Alternatively or additionally, a single value of first and/or second fuzzy set may be located at a locus 666 on first range 612 and/or second range 624, where a probability of membership may be taken by evaluation of first membership function 608 and/or second membership function 620 at that range point. A probability at 628 and/or 662 may be compared to a threshold 640 to determine whether a positive match is indicated. Threshold 640 may, in a non-limiting example, represent a degree of match between first fuzzy set 604 and second fuzzy set 616, and/or single values therein with each other or with either set, which is sufficient for purposes of the matching process; for instance, threshold may indicate a sufficient degree of overlap between an output from one or more machine-learning models, for combination to occur as described above. Alternatively or additionally, each threshold may be tuned by a machine-learning and/or statistical process, for instance and without limitation as described in further detail below.

Further referring to FIG. 6, an inference engine may be implemented according to input and/or output membership functions and/or linguistic variables. Continuing the example, an output linguistic variable may represent, without limitation, a score value. An inference engine may combine rules, such as: “if the cardio level is ‘high’ and the muscularity level is ‘medium’, the activity baseline is ‘medium-high’”—the degree to which a given input function membership matches a given rule may be determined by a triangular norm or “T-norm” of the rule or output membership function with the input membership function, such as min (a, b), product of a and b, drastic product of a and b, Hamacher product of a and b, or the like, satisfying the rules of commutativity (T(a, b)=T(b, a)), monotonicity: (T(a, b)≤T(c, d) if a≤c and b≤d), (associativity: T(a, T(b, c))=T(T(a, b), c)), and the requirement that the number 1 acts as an identity element. Combinations of rules (“and” or “or” combination of rule membership determinations) may be performed using any T-conorm, as represented by an inverted T symbol or “⊥,” such as max(a, b), probabilistic sum of a and b (a+b−a*b), bounded sum, and/or drastic T-conorm; any T-conorm may be used that satisfies the properties of commutativity: ⊥(a, b)=⊥(b, a), monotonicity: ⊥(a, b)≤⊥(c, d) if a≤c and b≤d, associativity: ⊥(a, ⊥(b, c))=≯(⊥(a, b), c), and identity element of 0. Alternatively or additionally T-conorm may be approximated by sum, as in a “product-sum” inference engine in which T-norm is product and T-conorm is sum. A final output score or other fuzzy inference output may be determined from an output membership function as described above using any suitable defuzzification process, including without limitation Mean of Max defuzzification, Centroid of Area/Center of Gravity defuzzification, Center Average defuzzification, Bisector of Area defuzzification, or the like. Alternatively or additionally, output rules may be replaced with functions according to the Takagi-Sugeno-King (TSK) fuzzy model.

It is to be noted that any one or more of the aspects and embodiments described herein may be conveniently implemented using one or more machines (e.g., one or more processors that are utilized as a user processor for an electronic document, one or more server devices, such as a document server, etc.) programmed according to the teachings of the present specification, as will be apparent to those of ordinary skill in the computer art. Appropriate software coding can readily be prepared by skilled programmers based on the teachings of the present disclosure, as will be apparent to those of ordinary skill in the software art. Aspects and implementations discussed above employing software and/or software modules may also include appropriate hardware for assisting in the implementation of the machine executable instructions of the software and/or software module.

Such software may be a computer program product that employs a machine-readable storage medium. A machine-readable storage medium may be any medium that is capable of storing and/or encoding a sequence of instructions for execution by a machine (e.g., a processor) and that causes the machine to perform any one of the methodologies and/or embodiments described herein. Examples of a machine-readable storage medium include, but are not limited to, a magnetic disk, an optical disc (e.g., CD, CD-R, DVD, DVD-R, etc.), a magneto-optical disk, a read-only memory “ROM” device, a random access memory “RAM” device, a magnetic card, an optical card, a solid-state memory device, an EPROM, an EEPROM, and any combinations thereof. A machine-readable medium, as used herein, is intended to include a single medium as well as a collection of physically separate media, such as, for example, a collection of compact discs or one or more hard disk drives in combination with a computer memory. As used herein, a machine-readable storage medium does not include transitory forms of signal transmission.

Such software may also include information (e.g., data) carried as a data signal on a data carrier, such as a carrier wave. For example, machine-executable information may be included as a data-carrying signal embodied in a data carrier in which the signal encodes a sequence of instruction, or portion thereof, for execution by a machine (e.g., a processor) and any related information (e.g., data structures and data) that causes the machine to perform any one of the methodologies and/or embodiments described herein.

Examples of a processor include, but are not limited to, an electronic book reading device, a computer workstation, a terminal computer, a server computer, a handheld device (e.g., a tablet computer, a smartphone, etc.), a web appliance, a network router, a network switch, a network bridge, any machine capable of executing a sequence of instructions that specify an action to be taken by that machine, and any combinations thereof. In one example, a processor may include and/or be included in a kiosk.

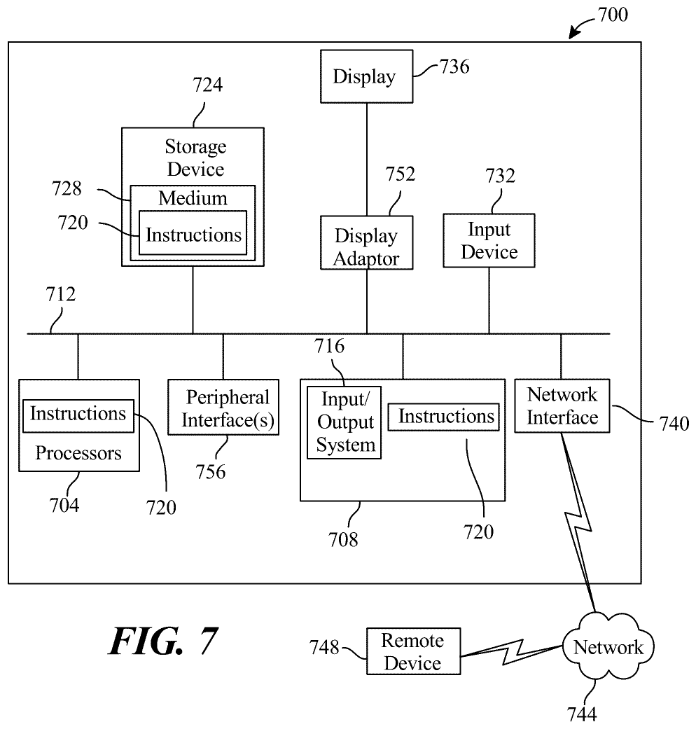

FIG. 7 shows a diagrammatic representation of one embodiment of a processor in the exemplary form of a computer system 700 within which a set of instructions for causing a control system to perform any one or more of the aspects and/or methodologies of the present disclosure may be executed. It is also contemplated that multiple processors may be utilized to implement a specially configured set of instructions for causing one or more of the devices to perform any one or more of the aspects and/or methodologies of the present disclosure. Computer system 700 includes a processor 704 and a memory 708 that communicate with each other, and with other components, via a bus 712. Bus 712 may include any of several types of bus structures including, but not limited to, a memory bus, a memory controller, a peripheral bus, a local bus, and any combinations thereof, using any of a variety of bus architectures.

Processor 704 may include any suitable processor, such as without limitation a processor incorporating logical circuitry for performing arithmetic and logical open circuits, such as an arithmetic and logic unit (ALU), which may be regulated with a state machine and directed by open circuital inputs from memory and/or sensors; processor 704 may be organized according to Von Neumann and/or Harvard architecture as a non-limiting example. Processor 704 may include, incorporate, and/or be incorporated in, without limitation, a microcontroller, microprocessor, digital signal processor (DSP), Field Programmable Gate Array (FPGA), Complex Programmable Logic Device (CPLD), Graphical Processing Unit (GPU), general purpose GPU, Tensor Processing Unit (TPU), analog or mixed signal processor, Trusted Platform Module (TPM), a floating point unit (FPU), and/or system on a chip (SoC).

Memory 708 may include various components (e.g., machine-readable media) including, but not limited to, a random-access memory component, a read only component, and any combinations thereof. In one example, a basic input/output system 717 (BIOS), including basic routines that help to transfer information between elements within computer system 700, such as during start-up, may be stored in memory 708. Memory 708 may also include (e.g., stored on one or more machine-readable media) instructions (e.g., software) 720 embodying any one or more of the aspects and/or methodologies of the present disclosure. In another example, memory 708 may further include any number of program modules including, but not limited to, an operating system, one or more application programs, other program modules, program data, and any combinations thereof.

Computer system 700 may also include a storage device 724. Examples of a storage device (e.g., storage device 724) include, but are not limited to, a hard disk drive, a magnetic disk drive, an optical disc drive in combination with an optical medium, a solid-state memory device, and any combinations thereof. Storage device 724 may be connected to bus 712 by an appropriate interface (not shown). Example interfaces include, but are not limited to, SCSI, advanced technology attachment (ATA), serial ATA, universal serial bus (USB), IEEE 1394 (FIREWIRE), and any combinations thereof. In one example, storage device 724 (or one or more components thereof) may be removably interfaced with computer system 700 (e.g., via an external port connector (not shown)). Particularly, storage device 724 and an associated machine-readable medium 728 may provide nonvolatile and/or volatile storage of machine-readable instructions, data structures, program modules, and/or other data for computer system 700. In one example, software 720 may reside, completely or partially, within machine-readable medium 728. In another example, software 720 may reside, completely or partially, within processor 704.

Computer system 700 may also include an input device 732. In one example, a user of computer system 700 may enter commands and/or other information into computer system 700 via input device 732. Examples of an input device 732 include, but are not limited to, an alpha-numeric input device (e.g., a keyboard), a pointing device, a joystick, a gamepad, an audio input device (e.g., a microphone, a voice response system, etc.), a cursor control device (e.g., a mouse), a touchpad, an optical scanner, a video capture device (e.g., a still camera, a video camera), a touchscreen, and any combinations thereof. Input device 732 may be interfaced to bus 712 via any of a variety of interfaces (not shown) including, but not limited to, a serial interface, a parallel interface, a game port, a USB interface, a FIREWIRE interface, a direct interface to bus 712, and any combinations thereof. Input device 732 may include a touch screen interface that may be a part of or separate from display 737, discussed further below. Input device 732 may be utilized as a user selection device for selecting one or more graphical representations in a graphical interface as described above.

A user may also input commands and/or other information to computer system 700 via storage device 724 (e.g., a removable disk drive, a flash drive, etc.) and/or network interface device 740. A network interface device, such as network interface device 740, may be utilized for connecting computer system 700 to one or more of a variety of networks, such as network 744, and one or more remote devices 748 connected thereto. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two processors, and any combinations thereof. A network, such as network 744, may employ a wired and/or a wireless mode of communication. In general, any network topology may be used. Information (e.g., data, software 720, etc.) may be communicated to and/or from computer system 700 via network interface device 740.

Computer system 700 may further include a video display adapter 752 for communicating a displayable image to a display device, such as display device 736. Examples of a display device include, but are not limited to, a liquid crystal display (LCD), a cathode ray tube (CRT), a plasma display, a light emitting diode (LED) display, and any combinations thereof. Display adapter 752 and display device 736 may be utilized in combination with processor 704 to provide graphical representations of aspects of the present disclosure. In addition to a display device, computer system 700 may include one or more other peripheral output devices including, but not limited to, an audio speaker, a printer, and any combinations thereof. Such peripheral output devices may be connected to bus 712 via a peripheral interface 756. Examples of a peripheral interface include, but are not limited to, a serial port, a USB connection, a FIREWIRE connection, a parallel connection, and any combinations thereof.

The foregoing has been a detailed description of illustrative embodiments of the invention. Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments, what has been described herein is merely illustrative of the application of the principles of the present invention. Additionally, although particular methods herein may be illustrated and/or described as being performed in a specific order, the ordering is highly variable within ordinary skill to achieve methods, systems, and software according to the present disclosure. Accordingly, this description is meant to be taken only by way of example, and not to otherwise limit the scope of this invention.

Exemplary embodiments have been disclosed above and illustrated in the accompanying drawings. It will be understood by those skilled in the art that various changes, omissions and additions may be made to that which is specifically disclosed herein without departing from the spirit and scope of the present invention.

1. An apparatus for generating a circuit protocol for instituting a desired body mass index (BMI) change comprising a computing device configured to:
receive at least a BMI representation and a circuit record comprising at least a digestion mode;
generate, as a function of the circuit record, at least a change of mode, wherein generating the at least a change of mode further comprises:
retrieving an activity baseline, wherein the activity baseline comprises an intensity baseline, a cardio baseline and a muscularity baseline;
receiving training data containing a plurality of data entries containing a plurality of inputs containing functional elements correlated to a plurality of outputs containing the at least a BMI representation; and
training a machine learning model as a function of a machine learning algorithm and the training data;
calculate a desired BMI change as a function of the at least a BMI representation;
produce at least a change in mode as a function of the trained machine learning model, and the circuit record;
obtain an activity profile, wherein the activity profile comprises at least an adjacent motion;
identify a plurality of activity categories as a function of the activity profile, wherein identifying a plurality of activity categories further comprises:
an activity category classifier, wherein the activity category classifier is trained to categorize activities listed within the activity profile to categories of activities and add activity recommendations based on the categories of activities; and
compute a desired increase in activity as a function of the activity baseline;
establish an activity type as a function of a plurality of activity types, wherein identifying the activity type further comprises:
an activity type classifier; and
output the circuit protocol as a function of the at least a change in mode.

2. The apparatus of claim 1, wherein receiving the at least a BMI representation further comprises recording levels of a plurality of diabesity markers wherein diabesity markers comprises data related to diabesity.

3. The apparatus according to claim 2, wherein diabesity markers data is collected through wearable devices.

4. The apparatus according to claim 2, wherein diabesity markers data is collected through computer system monitoring.

5. The apparatus according to claim 2, wherein diabesity markers data is collected through biological testing.

6. The apparatus of claim 1, wherein calculating the desired BMI change further comprises calculating a distance between the at least a BMI representation and a BMI standard.

7. The apparatus according to claim 6, wherein calculating the distance between the at least a BMI representation and the BMI standard further comprises the computing device configured to:
represent the at least a BMI representation as a first vector;
represent the BMI standard as a second vector;
calculate a similarity between the first vector and the second vector; and
calculate the distance as a function of the similarity between the first vector and the second vector.

8. The apparatus of claim 6, wherein generating the at least a change of mode further comprises:
generate a mode standard as a function of the machine learning model and the BMI standard;
calculate a distance between the circuit record and the mode standard; and
output the at least a change of mode as a function of the distance.

9. The apparatus of claim 1, wherein outputting the circuit protocol further comprises:
receiving circuit classification training data correlating a plurality of data entries containing a plurality of inputs containing the at least a BMI representation to a plurality of outputs containing a plurality of BMI representation bins;
training, as a function of a circuit classification algorithm, a circuit classification model and the circuit classification training data;
classify at least a circuit from the circuit record to at least a bin of a plurality of bins, as a function of the circuit classification model and the circuit record;
select, a new circuit classified to the at least a bin, as a function of the at least a change of mode; and
output, using the circuit protocol, wherein the circuit protocol comprises the new circuit.

10. The apparatus of claim 1 wherein outputting the circuit protocol further comprises the computing device configured to:
receive circuit classification training data correlating a plurality of circuits to a plurality of bins;
classify at least a circuit from the circuit record to at least a bin of the plurality of bins, as a function of the circuit classification model and the circuit record;
select a new circuit classified to the at least a bin, as a function of the at least a change in mode; and
output the circuit protocol, wherein the circuit protocol comprises the new circuit.

11. A method of generating a circuit protocol for instituting a desired body mass index (BMI) change comprising a computing device configured to:
receiving, by a processor, at least a BMI representation and a circuit record comprising at least a digestion mode;
generating, by the processor, as a function of the circuit record, at least a change of mode, wherein generating the at least a change of mode further comprises:
retrieving an activity baseline, wherein the activity baseline comprises an intensity baseline, a cardio baseline and a muscularity baseline;
receiving training data containing a plurality of data entries containing a plurality of inputs containing functional elements correlated to a plurality of outputs containing the at least a BMI representation;
training a machine learning model as a function of a machine learning algorithm and the training data;
calculating, by the processor, a desired BMI change as a function of the at least a BMI representation;
producing, by the processor, at least a change in mode as a function of the trained machine learning model, and the circuit record;
obtaining, by the processor, an activity profile, wherein the activity profile comprises at least an adjacent motion;
identifying, by the processor, a plurality of activity categories as a function of the activity profile, wherein identifying a plurality of activity categories further comprises:
an activity category classifier, wherein the activity category classifier is trained to categorize activities listed within the activity profile to categories of activities and add activity recommendations based on the categories of activities; and
computing, by the processor, a desired increase in activity as a function of the activity baseline;
establishing, by the processor, an activity type as a function of a plurality of activity types, wherein identifying the activity type further comprises:
an activity type classifier; and
outputting, by the processor, the circuit protocol as a function of the at least a change in mode.

12. The method of claim 11, wherein receiving the at least a BMI representation further comprises recording levels of a plurality of diabesity markers, wherein diabesity markers comprises data related to diabesity.

13. The method according to claim 12, wherein diabesity markers data is collected through wearable devices.

14. The method according to claim 12, wherein diabesity markers data is collected through computer method monitoring.

15. The method according to claim 12, wherein diabesity markers data is collected through biological testing.

16. The method of claim 11, wherein calculating the desired BMI change further comprises calculating a distance between the at least a BMI representation and a BMI standard.

17. The method of claim 16, wherein calculating the distance between the at least a BMI representation and the BMI standard further comprises the computing device configured to:
represent the at least a BMI representation as a first vector;
represent the BMI standard as a second vector;
calculate a similarity between the first vector and the second vector; and
calculate the distance as a function of the similarity between the first vector and the second vector.

18. The method of claim 16, wherein generating the at least a change of mode further comprises:
generate a mode standard as a function of the machine learning model and the BMI standard;
calculate a distance between the circuit record and the mode standard; and
output the at least a change of mode as a function of the distance.

19. The method of claim 11, wherein outputting the circuit protocol further comprises:
receiving circuit classification training data correlating a plurality of data entries containing a plurality of inputs containing the at least a BMI representation to a plurality of outputs containing a plurality of BMI representation bins;
training, as a function of a circuit classification algorithm, a circuit classification model and the circuit classification training data;
classify at least a circuit from the circuit record to at least a bin of a plurality of bins, as a function of the circuit classification model and the circuit record;
select, a new circuit classified to the at least a bin, as a function of the at least a change of mode; and
output, using the circuit protocol, wherein the circuit protocol comprises the new circuit.

20. The method of claim 11 wherein outputting the circuit protocol further comprises the computing device configured to:
receiving, by a processor, circuit classification training data correlating a plurality of circuits to a plurality of bins;
classifying, by the processor, at least a circuit from the circuit record to at least a bin of the plurality of bins, as a function of the circuit classification model and the circuit record;
selecting, by the processor, a new circuit classified to the at least a bin, as a function of the at least a change in mode; and
outputting, by the processor, the circuit protocol, wherein the circuit protocol comprises the new circuit.
